(19) United States
(12) Patent Application Publication
(10) Pub. No.: US 2021/0349035 A1
(43) Pub. Date: Nov. 11, 2021

(54) PATHOGEN MONITORING

(71) Applicant: Platypus Technologies, LLC, Madison, WI (US)

(72) Inventors: Marco A. Bedolla Pantoja, Madison, WI (US); Nicholas L. Abbott, Madison, WI (US)

(21) Appl. No.: 17/314,834

(22) Filed: May 7, 2021

Related U.S. Application Data
(60) Provisional application No. 63/021,375, filed on May 7, 2020.

(51) Int. Cl. G01N 21/78; G01N 33/569; A62B 9/00; A62B 18/08

(52) U.S. Cl. CPC G01N 21/78; G01N 33/56983; A62B 9/006; A41D 13/11; G01N 2333/165; G01N 2469/10; G01N 2800/26; A62B 18/08

(57) ABSTRACT

The present invention relates to the field of detection of analytes, and in particular to detection of viruses, cells, bacteria, and lipid-membrane containing organisms in respiratory droplets using a liquid crystal detection device positioned in a breathing barrier, on the skin or inside a device for collection of air or aerosols.

### CROSS-REFERENCE TO RELATED APPLICATIONS

This application claims the benefit of U.S. Provisional Application 63/021,375, filed May 7, 2020, the entire contents of which are incorporated herein by reference.

### FIELD OF THE INVENTION

The present invention relates to the field of detection of analytes, and in particular to detection of viruses, cells, bacteria, and lipid-membrane containing organisms in respiratory droplets using a liquid crystal detection device positioned in a breathing barrier, on the skin or inside a device for collection of air or aerosols.

### BACKGROUND OF THE INVENTION

Coronaviruses are a family of viruses that can cause illnesses such as the common cold, severe acute respiratory syndrome (SARS) and Middle East respiratory syndrome (MERS). In 2019, a new coronavirus was identified as the cause of a disease outbreak that originated in China. The virus is now known as the severe acute respiratory syndrome coronavirus 2 (SARS-CoV-2). The disease it causes is called coronavirus disease 2019 (COVID-19). Cases of COVID-19 have been reported around the world and WHO declared a global pandemic in March 2020.

Signs and symptoms of COVID-19 may appear two to 14 days after exposure and can include: fever; cough; and shortness of breath or difficulty breathing. Other symptoms can include: tiredness; aches; runny nose; and sore throat. The severity of COVID-19 symptoms can range from very mild to severe. Some people have no symptoms. People with no symptoms can still transmit the virus to other people, and thus promote spread of COVID-19. This makes assessment and control of the spread of COVID-19 difficult to manage and control based on strategies that involving monitoring of symptoms. People who are older or have existing chronic medical conditions, such as heart or lung disease or diabetes, may be at higher risk of serious illness.

What is needed in the art are methods for monitoring individuals and environments for infection by SARS-CoV-2 and other respiratory pathogens.

### SUMMARY OF THE INVENTION

The present invention relates to the field of detection of analytes, and in particular to detection of viruses, cells, bacteria, and lipid-membrane containing organisms in respiratory droplets using a liquid crystal detection device positioned in a breathing barrier, on the skin or inside a device for collection of air or aerosols.

In some preferred embodiments, the present invention provides a device for detecting biological entities comprising a lipid membrane comprising: a breathing barrier; and a liquid crystal detection unit comprising mesogens; wherein the liquid crystal detection unit is positioned within the breathing barrier so that when the breathing barrier is placed on a subject in the act of breathing the breath of the user passes through or over the liquid crystal detection unit.

In some preferred embodiments, the breathing barrier is selected from the group consisting of a respirator and a mask. In some preferred embodiments, the respirator is selected from the group consisting of an N95 respirator and a KN95 respirator. In some preferred embodiments, the mask is selected from the group consisting of a surgical mask and a cloth mask. In some preferred embodiments, the liquid crystal detection unit comprises a liquid crystal formed from mesogens disposed on the surface of a first substrate. In some preferred embodiments, the liquid crystal detection unit further comprises a second substrate positioned opposite to the first substrate to form a cell having a gap between the second substrate and the mesogens disposed on the first substrate. In some preferred embodiments, at least a portion of the breath of the user passes through the gap. In some preferred embodiments, the gap is an air gap.

In some preferred embodiments, the first substrate is selected from the group consisting of metal films, glass, silicon, diamond and polymeric materials. In some preferred embodiments, the polymeric materials are selected from the group consisting of polyurethane, PDMS, polyimide, polystyrene, polycarbonate and polyisocyanoacrylate. In some preferred embodiments, the second substrate is selected from the group consisting of metal films, glass, silicon, diamond and polymeric materials. In some preferred embodiments, the polymeric materials are selected from the group consisting of polyurethane, PDMS, polyimide, polystyrene, polycarbonate and polyisocyanoacrylate. In some preferred embodiments, the mesogens are selected from the group consisting of E7, MLC, 5CB (4-n-pentyl-4′-cyanobiphenyl), 8CB (4-cyano-4′octylbiphenyl), BL093, TL 216, ZLI 5800, MLC 6613, and MBBA ((p-methoxybenzylidene)-p-butylaniline) and combinations thereof.

In some preferred embodiments, the liquid crystal detection unit comprises one or more recognition moieties. In some preferred embodiments, the breath of the user forms an aqueous/liquid crystal interface and the recognition moieties are positioned at the aqueous/liquid crystal interface. In some preferred embodiments, the one or more recognition moieties are selected from the group consisting of antigen binding molecules, aptamers, and carbohydrates.

In some preferred embodiments, the present invention provides a method of detecting the presence of a biological entity comprising a lipid membrane in respiratory droplets in (or aerosols from) the breath of a subject comprising: providing a device as described above; positioning the device on the subject so that when the subject breathes at least a portion of breath containing respiratory droplets of the subject passes through the liquid crystal detection unit; and monitoring the liquid crystal detection device for changes in the mesogens after or during exposure of the mesogens to the breath of the subject, wherein the change in the mesogens is indicative of the presence or absence of a biological entity comprising a lipid membrane in respiratory droplets.

In some preferred embodiments, the change in the mesogens is selected from the group consisting of a change in color, a change in texture, a change in tilt, and homeotropic orientation. In some preferred embodiments, the change in mesogens is detected by a method selected from the group consisting of visual detection, optical detection, spectroscopy, light transmission, and electrical detection. In some preferred embodiments, the biological entity comprising a lipid membrane is selected from the group consisting of mycoplasmas, bacteria and viruses. In some preferred embodiments, the virus is selected from the group consisting of the following families: Adenoviridae, Arenaviridae, Astroviridae, Birnaviridae, Bunyaviridae, Caliciviridae, Circoviridae, Coronaviridae, Filoviridae, Flaviviridae, Hepadnaviridae, Herpesviridae, Iridoviridae, Filoviridae, Orthomyxoviridae, Papovaviridae, Paramyxoviridae, Parvoviridae, Picornaviridae, Poxviridae, Reoviridae, Retroviridae, Rhabdoviridae, Togaviridae, Badnavirus, Bromoviridae, Comoviridae, Geminiviridae, Partitiviridae, Potyviridae, Sequiviridae, and Tombusviridae. In some preferred embodiments, the virus is a member of the family Coronaviridae. In some preferred embodiments, the virus is SARS-CoV-2. In some preferred embodiments, the virus is a member of the family Orthomyxoviridae. In some preferred embodiments, the virus in an influenza A or influenza B virus. In some preferred embodiments, the method further comprises obtaining an image of the mesogens in the LC detection device and using an artificial intelligence algorithm to analyze the image to determine if there has been exposure to an entity comprising a biological membrane.

In some preferred embodiments, the present invention provides methods for detecting the presence of a biological entity comprising a lipid membrane in an environment inhabited by subjects comprising: collecting air from an environment inhabited by subjects; generating aqueous aerosol particles from the collected air; applying the aerosol particles to a liquid crystal detection unit comprising mesogens; and monitoring the liquid crystal detection device for changes in the mesogens after or during exposure of the mesogens to the aqueous aerosol particles, wherein the change in the mesogens is indicative of the presence or absence of a biological entity comprising a lipid membrane in the aerosol particles.

In some preferred embodiments, the change in the mesogens is selected from the group consisting of a change in color, a change in texture, a change in tilt, and homeotropic orientation. In some preferred embodiments, the change in mesogens is detected by a method selected from the group consisting of visual detection, optical detection, spectroscopy, light transmission, and electrical detection. In some preferred embodiments, the biological entity comprising a lipid membrane is selected from the group consisting of mycoplasmas, bacteria and viruses. In some preferred embodiments, the virus is selected from the group consisting of the following families: Adenoviridae, Arenaviridae, Astroviridae, Birnaviridae, Bunyaviridae, Caliciviridae, Circoviridae, Coronaviridae, Filoviridae, Flaviviridae, Hepadnaviridae, Herpesviridae, Iridoviridae, Filoviridae, Orthomyxoviridae, Papovaviridae, Paramyxoviridae, Parvoviridae, Picornaviridae, Poxviridae, Reoviridae, Retroviridae, Rhabdoviridae, Togaviridae, Badnavirus, Bromoviridae, Comoviridae, Geminiviridae, Partitiviridae, Potyviridae, Sequiviridae, and Tombusviridae. In some preferred embodiments, the virus is a member of the family Coronaviridae. In some preferred embodiments, the virus is SARS-CoV-2. In some preferred embodiments, the virus is a member of the family Orthomyxoviridae. In some preferred embodiments, the virus in an influenza A or influenza B virus. In some preferred embodiments, the methods further comprise obtaining an image of the mesogens in the LC detection device and using an artificial intelligence algorithm to analyze the image to determine if there has been exposure to an entity comprising a biological membrane.

In some preferred embodiments, the present invention provides methods for detecting the presence of a biological entity comprising a lipid membrane in an environment inhabited by subjects comprising: collecting air comprising aqueous aerosol particles from an environment inhabited by subjects; contacting a liquid crystal detection unit comprising mesogens with the aqueous aerosol particles; and monitoring the liquid crystal detection device for changes in the mesogens after or during exposure of the mesogens to the aqueous aerosol particles, wherein the change in the mesogens is indicative of the presence or absence of a biological entity comprising a lipid membrane in the aerosol particles.

In some preferred embodiments, the change in the mesogens is selected from the group consisting of a change in color, a change in texture, a change in tilt, and homeotropic orientation. In some preferred embodiments, the change in mesogens is detected by a method selected from the group consisting of visual detection, optical detection, spectroscopy, light transmission, and electrical detection. In some preferred embodiments, the biological entity comprising a lipid membrane is selected from the group consisting of mycoplasmas, bacteria and viruses. In some preferred embodiments, the virus is selected from the group consisting of the following families: Adenoviridae, Arenaviridae, Astroviridae, Birnaviridae, Bunyaviridae, Caliciviridae, Circoviridae, Coronaviridae, Filoviridae, Flaviviridae, Hepadnaviridae, Herpesviridae, Iridoviridae, Filoviridae, Orthomyxoviridae, Papovaviridae, Paramyxoviridae, Parvoviridae, Picornaviridae, Poxviridae, Reoviridae, Retroviridae, Rhabdoviridae, Togaviridae, Badnavirus, Bromoviridae, Comoviridae, Geminiviridae, Partitiviridae, Potyviridae, Sequiviridae, and Tombusviridae. In some preferred embodiments, the virus is a member of the family Coronaviridae. In some preferred embodiments, the virus is SARS-CoV-2. In some preferred embodiments, the virus is a member of the family Orthomyxoviridae. In some preferred embodiments, the virus in an influenza A or influenza B virus. In some preferred embodiments, the methods further comprise obtaining an image of the mesogens in the LC detection device and using an artificial intelligence algorithm to analyze the image to determine if there has been exposure to an entity comprising a biological membrane.

In some preferred embodiments, the present invention provides systems for detecting biological entities comprising a lipid membrane comprising: a liquid crystal detection unit comprising mesogens disposed on the surface of a first substrate and a second substrate positioned opposite to the first substrate to form a cell having a gap between the second substrate and the mesogens disposed on the first substrate; and an air and/or aerosol collection unit.

In some preferred embodiments, the liquid crystal detection unit further comprises an adhesive backing. In some preferred embodiments, the adhesive backing is compatible with attachment of the liquid crystal detection unit to the skin. In some preferred embodiments, the adhesive backing is compatible with attachment of the liquid crystal detection unit to the aerosol collection unit. In some preferred embodiments, the air/aerosol collection unit is a breathing barrier is selected from the group consisting of a respirator and a mask. In some preferred embodiments, the respirator is selected from the group consisting of an N95 respirator and a KN95 respirator. In some preferred embodiments, the mask is selected from the group consisting of a surgical mask and a cloth mask.

In other preferred embodiments, the air/aerosol collection unit samples air from an atmosphere.

In some preferred embodiments, the first substrate is selected from the group consisting of metal films, glass, silicon, diamond and polymeric materials. In some preferred embodiments, the polymeric materials are selected from the group consisting of polyurethane, PDMS, polyimide, polystyrene, polycarbonate and polyisocyanoacrylate. In some preferred embodiments, the second substrate is selected from the group consisting of metal films, glass, silicon, diamond and polymeric materials. In some preferred embodiments, the polymeric materials are selected from the group consisting of polyurethane, PDMS, polyimide, polystyrene, polycarbonate and polyisocyanoacrylate. In some preferred embodiments, the mesogens are selected from the group consisting of E7, MLC, 5CB (4-n-pentyl-4′-cyanobiphenyl), 8CB (4-cyano-4′octylbiphenyl), BL093, TL 216, ZLI 5800, MLC 6613, and MBBA ((p-methoxybenzylidene)-p-butylaniline) and combinations thereof.

In some preferred embodiments, the liquid crystal detection unit comprises one or more recognition moieties. In some preferred embodiments, contacting of aerosol particles with the liquid crystal forms an aqueous/liquid crystal interface and the recognition moieties are positioned at the aqueous/liquid crystal interface. In some preferred embodiments, the one or more recognition moieties are selected from the group consisting of antigen binding molecules, aptamers, and carbohydrates.

In some preferred embodiments, the present invention provides methods of detecting the presence of a biological entity comprising a lipid membrane comprising: providing a system as described above; exposing the liquid crystal detection device to an air and/or aerosol source suspected of containing the biological entity comprising a lipid membrane; and monitoring the liquid crystal detection device for changes in the mesogens after or during exposure of the mesogens to the air source, wherein the change in the mesogens is indicative of the presence or absence of a biological entity comprising a lipid membrane in the air source.

In some preferred embodiments, the change in the mesogens is selected from the group consisting of a change in color, a change in texture, a change in tilt, and homeotropic orientation. In some preferred embodiments, the change in mesogens is detected by a method selected from the group consisting of visual detection, optical detection, spectroscopy, light transmission, and electrical detection. In some preferred embodiments, the biological entity comprising a lipid membrane is selected from the group consisting of mycoplasmas, bacteria and viruses. In some preferred embodiments, the virus is selected from the group consisting of the following families: Adenoviridae, Arenaviridae, Astroviridae, Birnaviridae, Bunyaviridae, Caliciviridae, Circoviridae, Coronaviridae, Filoviridae, Flaviviridae, Hepadnaviridae, Herpesviridae, Iridoviridae, Filoviridae, Orthomyxoviridae, Papovaviridae, Paramyxoviridae, Parvoviridae, Picornaviridae, Poxviridae, Reoviridae, Retroviridae, Rhabdoviridae, Togaviridae, Badnavirus, Bromoviridae, Comoviridae, Geminiviridae, Partitiviridae, Potyviridae, Sequiviridae, and Tombusviridae. In some preferred embodiments, the virus is a member of the family Coronaviridae. In some preferred embodiments, the virus is SARS-CoV-2. In some preferred embodiments, the virus is a member of the family Orthomyxoviridae. In some preferred embodiments, the virus in an influenza A or influenza B virus.

In some preferred embodiments, the methods further comprise obtaining an image of the mesogens in the LC detection device and using an artificial intelligence algorithm to analyze the image to determine if there has been exposure to an entity comprising a biological membrane.

In some preferred embodiments, the air source is the breath of a subject. In some preferred embodiments, the liquid crustal detection unit is positioned on the skin of a subject and beneath a breathing barrier. In some preferred embodiments, the liquid crustal detection unit is positioned on a breathing barrier. In some preferred embodiments, the air source is air collected from an environment inhabited by one or more subjects. In some preferred embodiments, the environment is a room inhabited by one or more subjects.

### DESCRIPTION OF THE FIGURES

FIG. 1 is an illustration of an LC (Liquid Crystal)-based indicator attached to the interior of a face mask. The LC-based indicator changes color if viral particles (e.g. from COVID-19) have been detected.

FIG. 2A-C provides illustrations of: (a) an example of LC film supported on a solid substrate. When observed with cross-polarized light, the LC film appears dark; b) an example of LC-based indicator attached to face mask; and (c) a user wearing face mask containing LC indicator.

FIG. 3A-B provides an example of an LC film exposed to respiratory aerosol. Information regarding the contents of the respiratory aerosol can be obtained by analyzing the optical signature of the LC film.

FIG. 4 provides an illustration of film of liquid crystal enclosed between two plates separated by a defined distance such that a gap is formed between the two plates. Moist air, from breath flows through the gap between the two plates and deposits at the LC-air interface, causing an alignment transition in the LC. If a pathogen (virus or bacteria) is present in the respiratory droplets, the LC will exhibit a distinct alignment at the LC-aqueous interface.

FIG. 5A-D provides data obtained from exposure of an LC film to model viral particles in an aqueous aerosol.

FIG. 6A-B provides data obtained from exposure of an LC film to peppermint oil in an aqueous aerosol.

FIG. 7 is a photograph of a liquid crystal sensor utilized in Example 3.

FIG. 8 provides a photograph of representative LC films before exposure.

FIG. 9 provides a photograph of representative 25 μm thick E7 planar LC films 1 to 2 hours after exposure to each the four aerosol solutions.

FIG. 10 provides a photograph of representative 50 μm thick E7 planar LC films 1 to 2 hours after exposure to each the four aerosol solutions.

FIG. 11 provides a photograph of representative 25 μm thick E7 homeotropic LC films 1 to 2 hours after exposure to each the four aerosol solutions.

FIG. 12 provides a photograph of representative 50 μm thick E7 homeotropic LC films 1 to 2 hours after exposure to each the four aerosol solutions.

FIG. 13 provides a graphical summary of intensity for exposure of the different LC films to each of the four different solutions.

### DEFINITIONS

As used herein, the term “recognition moiety” refers to a composition of matter that interacts with an analyte of interest in either a covalent or noncovalent manner.

As used herein, the term “virus recognition moiety” refers to any composition of matter that binds specifically to a virus. Examples of “virus recognition moieties” include, but are not limited to antigen binding proteins and nucleic acid aptamers.

The term “substrate” refers to a composition that serves as a base for another composition such as recognition moiety. Examples of substrates include, but are not limited to, silicon surfaces, glass surfaces, polymer surfaces, glass beads, magnetic beads, agarose beads, etc.

As used herein, the term “ligand” refers to any molecule that binds to or can be bound by another molecule. A ligand is any ion, molecule, molecular group, or other substance that binds to another entity to form a larger complex. Examples of ligands include, but are not limited to, peptides, carbohydrates, nucleic acids, antibodies, or any molecules that bind to receptors.

As used herein, the term “homeotropic director” refers to a topographical feature (e.g., a nanostructure or homeotropic orienting polyimide) of a substrate that homeotropically orients a liquid crystal.

As used herein, the term “pathogen” refers to disease causing organisms, microorganisms, or agents including, but not limited to, viruses, bacteria, parasites (including, but not limited to, organisms within the phyla Protozoa, Platyhelminthes, Aschelminithes, Acanthocephala, and Arthropoda), fungi, and prions.

As used herein, the term “bacteria” and “bacterium” refer to all prokaryotic organisms, including those within all of the phyla in the Kingdom Procaryotae. It is intended that the term encompass all microorganisms considered to be bacteria including Mycoplasma, Chlamydia, Actinomyces, Streptomyces, and Rickettsia. All forms of bacteria are included within this definition including cocci, bacilli, spirochetes, spheroplasts, protoplasts, etc. “Gram negative” and “gram positive” refer to staining patterns obtained with the Gram-staining process which is well known in the art (See e.g., Finegold and Martin, Diagnostic Microbiology, 6th Ed. (1982), CV Mosby St. Louis, pp 13-15).

As used herein, the term “lipid membrane” refers to, in its broadest sense, a thin sheet or layer comprising lipid molecules. It is intended that the term encompass all “biomembranes” (i.e., any organic membrane including, but not limited to, plasma membranes, nuclear membranes, organelle membranes, and synthetic membranes). Typically, membranes are composed of lipids, proteins, glycolipids, steroids, sterol and/or other components. As used herein, the term “membrane fragment” refers to any portion or piece of a membrane.

As used herein, the term “lipid” refers to a variety of compounds that are characterized by their solubility in organic solvents. Such compounds include, but are not limited to, fats, waxes, steroids, sterols, glycolipids, glycosphingolipids (including gangliosides), phospholipids, terpenes, fat-soluble vitamins, prostaglandins, carotenes, and chlorophylls. As used herein, the phrase “lipid-based materials” refers to any material that contains lipids.

As used herein, the term “secondary binding agent” refer to a molecule or collection of molecules that binds to one of an analyte-recognition moiety complex. It is contemplated that secondary binding agents are useful for amplifying the signal resulting from analyte-recognition moiety binding.

As used herein, the term “column media” refers to media used to fill a chromatography column, such as cationic exchange media, anionic exchange media, and immunoaffinity column media.

As used herein, the term “detection region” refers to a discrete area on substrate that is designated for detection of an analyte (e.g., a virus of interest) in a sample.

As used herein, the term “immobilization” refers to the attachment or entrapment, either chemically or otherwise, of a material to another entity (e.g., to a mesogen, interface or substrate) in a manner that restricts the movement of the material.

As used herein, the terms “material” and “materials” refer to, in their broadest sense, any composition of matter.

As used herein the term “antigen binding protein” refers to a glycoprotein evoked in an animal by an immunogen (antigen) and to proteins derived from such glycoprotein (e.g., single chain antibodies and F(ab′)2, Fab′ and Fab fragments). An antibody demonstrates specificity to the immunogen, or, more specifically, to one or more epitopes contained in the immunogen. Native antibody comprises at least two light polypeptide chains and at least two heavy polypeptide chains. Each of the heavy and light polypeptide chains contains at the amino terminal portion of the polypeptide chain a variable region (i.e., VH and VL respectively), which contains a binding domain that interacts with antigen. Each of the heavy and light polypeptide chains also comprises a constant region of the polypeptide chains (generally the carboxy terminal portion) which may mediate the binding of the immunoglobulin to host tissues or factors influencing various cells of the immune system, some phagocytic cells and the first component (C1q) of the classical complement system. The constant region of the light chains is referred to as the “CL region,” and the constant region of the heavy chain is referred to as the “CH region.” The constant region of the heavy chain comprises a CH1 region, a CH2 region, and a CH3 region. A portion of the heavy chain between the CH1 and CH2 regions is referred to as the hinge region (i.e., the “H region”). The constant region of the heavy chain of the cell surface form of an antibody further comprises a spacer-transmembranal region (M1) and a cytoplasmic region (M2) of the membrane carboxy terminus. The secreted form of an antibody generally lacks the M1 and M2 regions.

As used herein, the term “selective binding” refers to the binding of one material to another in a manner dependent upon the presence of a particular molecular structure (i.e., specific binding). For example, an immunoglobulin will selectively bind an antigen that contains the chemical structures complementary to the ligand binding site(s) of the immunoglobulin. This is in contrast to “non-selective binding,” whereby interactions are arbitrary and not based on structural compatibilities of the molecules.

As used herein, the term “polymerization” encompasses any process that results in the conversion of small molecular monomers into larger molecules consisting of repeated units. Typically, polymerization involves chemical crosslinking of monomers to one another.

As used herein, the term “antigen” refers to any molecule or molecular group that is recognized by at least one antibody. By definition, an antigen must contain at least one epitope (i.e., the specific biochemical unit capable of being recognized by the antibody). The term “immunogen” refers to any molecule, compound, or aggregate that induces the production of antibodies. By definition, an immunogen must contain at least one epitope (i.e., the specific biochemical unit capable of causing an immune response).

As used herein, the terms “home testing” and “point of care testing” refer to testing that occurs outside of a laboratory environment. Such testing can occur indoors or outdoors at, for example, a private residence, a place of business, public or private land, in a vehicle, as well as at the patient's bedside.

As used herein, the term “virus” refers to minute infectious agents, which with certain exceptions, are not observable by light microscopy, lack independent metabolism, and are able to replicate only within a living host cell. The individual particles (i.e., virions) consist of nucleic acid and a protein shell or coat; some virions also have a lipid containing membrane. The term “virus” encompasses all types of viruses, including animal, plant, phage, and other viruses.

As used herein, term “nanostructures” refers to microscopic structures, typically measured on a nanometer scale. Such structures include various three-dimensional assemblies, including, but not limited to, liposomes, films, multilayers, braided, lamellar, helical, tubular, and fiber-like shapes, and combinations thereof. Such structures can, in some embodiments, exist as solvated polymers in aggregate forms such as rods and coils. Such structures can also be formed from inorganic materials, such as prepared by the physical deposition of a gold film onto the surface of a solid, proteins immobilized on surfaces that have been mechanically rubbed, and polymeric materials that have been molded or imprinted with topography by using a silicon template prepared by electron beam lithography.

As used herein, the terms “self-assembling monomers” and “lipid monomers” refer to molecules that spontaneously associate to form molecular assemblies. In one sense, this can refer to surfactant molecules that associate to form surfactant molecular assemblies. The term “self-assembling monomers” includes single molecules (e.g., a single lipid molecule) and small molecular assemblies (e.g., polymerized lipids), whereby the individual small molecular assemblies can be further aggregated (e.g., assembled and polymerized) into larger molecular assemblies.

As used herein, the term “linker” or “spacer molecule” refers to material that links one entity to another. In one sense, a molecule or molecular group can be a linker that is covalent attached two or more other molecules (e.g., linking a ligand to a self-assembling monomer).

As used herein, the term “bond” refers to the linkage between atoms in molecules and between ions and molecules in crystals. The term “single bond” refers to a bond with two electrons occupying the bonding orbital. Single bonds between atoms in molecular notations are represented by a single line drawn between two atoms (e.g., C—C). The term “double bond” refers to a bond that shares two electron pairs. Double bonds are stronger than single bonds and are more reactive. The term “triple bond” refers to the sharing of three electron pairs. As used herein, the term “ene-yne” refers to alternating double and triple bonds. As used herein the terms “amine bond,” “thiol bond,” and “aldehyde bond” refer to any bond formed between an amine group (i.e., a chemical group derived from ammonia by replacement of one or more of its hydrogen atoms by hydrocarbon groups), a thiol group (i.e., sulfur analogs of alcohols), and an aldehyde group (i.e., the chemical group —CHO joined directly onto another carbon atom), respectively, and another atom or molecule.

As used herein, the term “covalent bond” refers to the linkage of two atoms by the sharing of two electrons, one contributed by each of the atoms.

As used herein, the term “spectrum” refers to the distribution of light energies arranged in order of wavelength.

As used the term “visible spectrum” refers to light radiation that contains wavelengths from approximately 360 nm to approximately 800 nm.

As used herein, the term “substrate” refers to a solid object or surface upon which another material is layered or attached. Solid supports include, but are not limited to, glass, metals, gels, and filter paper, among others.

As used herein, the terms “array” and “patterned array” refer to an arrangement of elements (i.e., entities) into a material or device. For example, combining several types of ligand binding molecules (e.g., antibodies or nucleic acids) into an analyte-detecting device, would constitute an array.

As used herein, the term “in situ” refers to processes, events, objects, or information that are present or take place within the context of their natural environment.

As used herein, the term “sample” is used in its broadest sense. In one sense it can refer to a biopolymeric material. In another sense, it is meant to include a specimen or culture obtained from any source, as well as biological and environmental samples.

Biological samples may be obtained from animals (including humans) and encompass fluids, solids, tissues, and gases. Biological samples include blood products, such as plasma, serum and the like. Environmental samples include environmental material such as surface matter, soil, water, crystals and industrial samples. These examples are not to be construed as limiting the sample types applicable to the present invention.

As used herein, the term “liquid crystal” refers to a thermodynamic stable phase characterized by anisotropy of properties without the existence of a three-dimensional crystal lattice, generally lying in the temperature range between the solid and isotropic liquid phase.

As used herein, the term “mesogen” refers compounds that form liquid crystals, and in particular rigid rodlike or disclike molecules that are components of liquid crystalline materials.

As used herein, “thermotropic liquid crystal” refers to liquid crystals that result from the melting of mesogenic solids due to an increase in temperature. Both pure substances and mixtures form thermotropic liquid crystals.

“Lyotropic,” as used herein, refers to molecules that form phases with orientational and/or positional order in a solvent. Lyotropic liquid crystals can be formed using amphiphilic molecules (e.g., sodium laurate, phosphatidylethanolamine, lecithin). The solvent can be water.

As used herein, the term “heterogenous surface” refers to a surface that orients liquid crystals in at least two separate planes or directions, such as across a gradient.

As used herein, “nematic” refers to liquid crystals in which the long axes of the molecules remain substantially parallel, but the positions of the centers of mass are randomly distributed. Nematic liquid crystals can be substantially oriented by a nearby surface.

“Chiral nematic,” as used herein refers to liquid crystals in which the mesogens are optically active. Instead of the director being held locally constant as is the case for nematics, the director rotates in a helical fashion throughout the sample. Chiral nematic crystals show a strong optical activity that is much higher than can be explained on the bases of the rotatory power of the individual mesogens. When light equal in wavelength to the pitch of the director impinges on the liquid crystal, the director acts like a diffraction grating, reflecting most and sometimes all of the light incident on it. If white light is incident on such a material, only one color of light is reflected and it is circularly polarized. This phenomenon is known as selective reflection and is responsible for the iridescent colors produced by chiral nematic crystals.

“Smectic,” as used herein refers to liquid crystals which are distinguished from “nematics” by the presence of a greater degree of positional order in addition to orientational order; the molecules spend more time in planes and layers than they do between these planes and layers. “Polar smectic” layers occur when the mesogens have permanent dipole moments. In the smectic A2 phase, for example, successive layers show anti ferroelectric order, with the direction of the permanent dipole alternating from layer to layer. If the molecule contains a permanent dipole moment transverse to the long molecular axis, then the chiral smectic phase is ferroelectric. A device utilizing this phase can be intrinsically bistable.

“Frustrated phases,” as used herein, refers to another class of phases formed by chiral molecules. These phases are not chiral, however, twist is introduced into the phase by an array of grain boundaries. A cubic lattice of defects (where the director is not defined) exist in a complicated, orientationally ordered twisted structure. The distance between these defects is hundreds of nanometers, so these phases reflect light just as crystals reflect x-rays.

“Discotic phases” are formed from molecules that are disc shaped rather than elongated. Usually these molecules have aromatic cores and six lateral substituents. If the molecules are chiral or a chiral dopant is added to a discotic liquid crystal, a chiral nematic discotic phase can form.

### DESCRIPTION OF THE INVENTION

The present invention relates to the field of detection of analytes, and in particular to detection of viruses, cells, bacteria, and lipid-membrane containing organisms in respiratory droplets using a liquid crystal detection device positioned in a breathing barrier, on the skin or inside a device for collection of air or aerosols.

Airborne diseases are caused by viruses or bacteria contained in respiratory aerosols emitted from infected individuals. Respiratory aerosols are generated when individuals breathe, talk, cough or sneeze. Examples of viral diseases that are transmitted via airborne respiratory droplets include measles, influenza, chickenpox, common cold, SARS, MERS, and COVID-19. Similarly, bacterial infections transmitted via airborne droplets include tuberculosis, legionnaires' disease and whooping cough. Devices and methods that detect airborne species involved in the transmission of disease find use in management of the spread of the disease.

The current reported shortages for testing kits for COVID-19 in the United States highlights the need for tests to identify individuals infected with airborne diseases. Even when infected individuals do not exhibit measurable symptoms, they transmit the disease via respiratory aerosols. Thus, methods to visually detect the presence of respiratory particles that indicate infection could be valuable in managing the spread of infectious disease.

Liquid crystal-based assay systems (LC assays) are described in U.S. Pat. Nos. 8,988,620; 9,816,147; 6,284,197; WO 01/61357; WO 01/61325; WO 99/63329; Gupta et al., Science 279:2077-2080 (1998); Seung-Ryeol Kim, Rahul R. Shah, and Nicholas L. Abbott; Orientations of Liquid Crystals on Mechanically Rubbed Films of Bovine Serum Albumin: A Possible Substrate for Biomolecular Assays Based on Liquid Crystals, Analytical Chemistry; 2000; 72(19); 4646-4653; Justin J. Skaife and Nicholas L. Abbott; Quantitative Interpretation of the Optical Textures of Liquid Crystals Caused by Specific Binding of Immunoglobulins to Surface-Bound Antigens, Langmuir; 2000; 16(7); 3529-3536; Vinay K. Gupta and Nicholas L. Abbott; Using Droplets of Nematic Liquid Crystal To Probe the Microscopic and Mesoscopic Structure of Organic Surfaces, Langmuir; 1999; 15(21); 7213-7223; all of which are incorporated herein by reference.

The present invention is based on the observation that liquid crystal (LC) films supported on surfaces containing enveloped viruses appear dark when viewed with cross-polarized light. This observation suggests that enveloped viruses, due to their lipid membrane, align the molecules of the LC in the orientation perpendicular to the supporting surface (i.e., homeotropic orientation). See, e.g., U.S. Pat. Nos. 8,988,620 and 9,816,147, each of which is incorporated herein by reference in its entirety. In contrast, water induces parallel alignment of the LC molecules, so LC films appear bright when in contact with a water or aqueous solution. Thus, it is contemplated that the presence of a virus or bacteria in an aqueous aerosol (such as respiratory droplets in the breath of a subject) deposited on an LC film will cause an alignment transition on the LC. This alignment transition is expressed as a characteristic change in the optical properties of the LC film. Thus, the methods of the present invention for detection of airborne disease agents comprising analyzing or monitoring the optical properties of LC films exposed to respiratory droplets to determine the pathogens in respiratory droplets and diagnose disease.

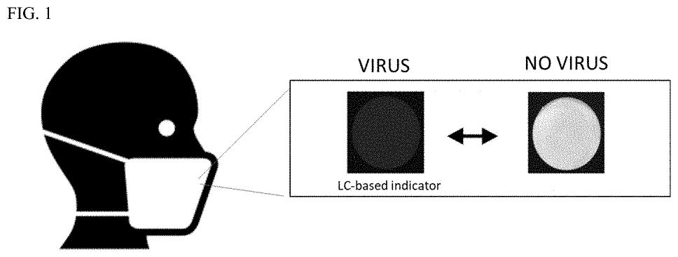

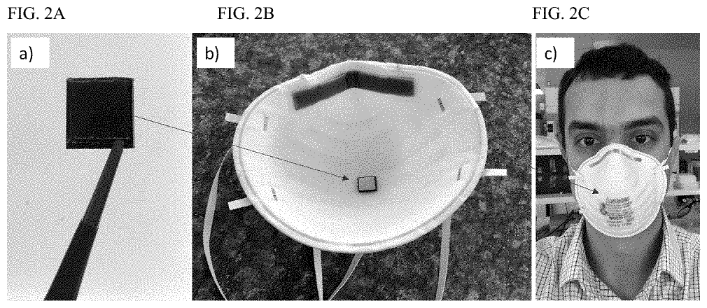

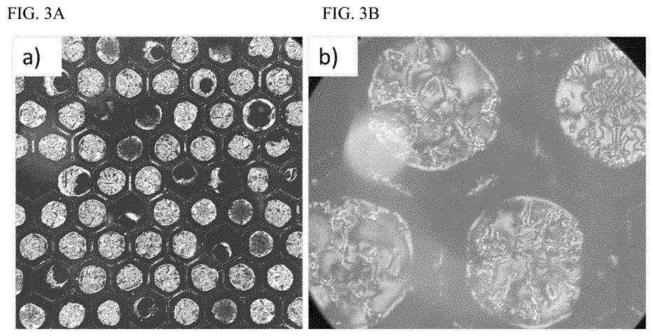

In some preferred embodiments, the present invention provides a device comprising a liquid crystal detection unit positioned in a breathing barrier. In some preferred embodiments, when the breathing barrier is placed on a subject in the act of breathing the breath of the user passes through or over the liquid crystal detection unit (LC detection unit). In some preferred embodiments, the liquid crystal detection unit produces an optical change due to the presence of a biological entity comprising a lipid membrane such as enveloped viruses or bacteria in respiratory droplets. In some preferred embodiments, the LC detection unit comprises a thin film of liquid crystal, formed from mesogens, supported on a solid substrate. The LC detection unit may preferably be designed to adhere to the interior side of breathing barriers such as face masks, respirators, surgical masks, or face shields. FIGS. 1 and 2 provides a schematic depiction and illustrations of a breathing barrier comprising an LC detection unit. FIG. 3 provides images of LC films exposed to a respiratory aerosol.

In some preferred embodiments, the subject will wear the breathing barrier containing the LC detection unit for a pre-defined time period. Then, the LC detection unit will be analyzed, either directly (with the naked eye) or using an optical device such as a microscope, a spectrometer, a camera, including smart phone camera, or a plate reader. By comparing the optical state of the indicator with a calibrated optical state, the user can determine the presence or absence of pathogens in respiratory droplets. In some preferred embodiments, machine learning methods are utilized to analyze the spatial features of the optical response and will be trained to identify spatial features that indicate the presence of respiratory droplets that contain an infectious pathogen. In some preferred embodiments, the image of the optical state of the LC will be transmitted to a computer where the image analysis will be performed.

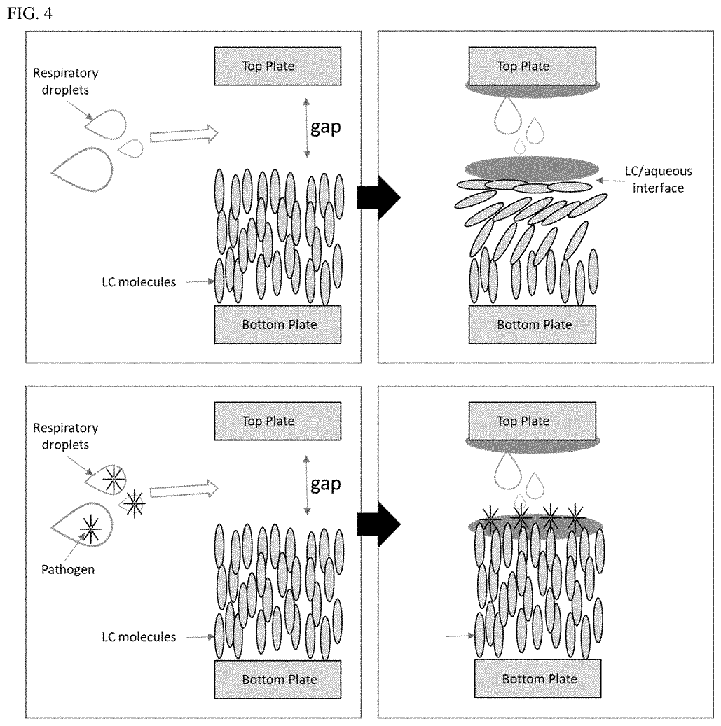

As exemplified in FIG. 4, in some preferred embodiments, the LC detection unit comprises a film of liquid crystal enclosed between two substrates (glass, silicon, polymer, etc.) separated by a defined distance such that a gap is formed between the two substrates. Moist air, from breath flows through the gap between the two plates and deposits at the LC-air interface, causing an alignment transition in the LC.

In some preferred embodiments, the interface of the liquid crystal film is decorated with recognition moieties that recognize specific biological entities within respiratory droplets and enrich the interface of the liquid crystal with those species.

In some embodiments, it is contemplated that the LC detectors have an adhesive backing and can be attached to the skin of a person in an area that lies beneath a cloth face covering or face mask. In some embodiments, it is contemplated that LC detectors can be incorporated inside device used for collecting aerosols (a “bioaerosol sampler”) and used for monitoring worker exposure in the context of industrial hygiene.

In some preferred embodiments, systems are provided that comprise a liquid crystal detection unit and an air collection unit or sampler. The systems are utilized to expose air expelled directly from a subject or that is sampled from an environment, such as a room, inhabited by one or more subjects, to the liquid crystal detection unit. The presence of an entity comprising a biological membrane may be detected as described above. In some preferred embodiments, the liquid crystal detection unit comprises an adhesive backing so that it may be placed, for example, on the skin of a subject beneath a breathing barrier or on the breathing barrier itself. In other preferred embodiments, the liquid crystal detection unit is attached to or positioned in an environmental aerosol or particulate sampler. Suitable samplers are known in the art and include, but are not limited to, those described in Verreault et al., Methods for Sampling of Airborne Viruses, Microbiol Mol Biol Rev. 2008 September; 72(3): 413-444, which is incorporated herein by reference in its entirety. In some preferred embodiments, the sampler is a cyclone sampler, a filter cassette sampler, an AGI-30 sampler, a swirling aerosol collector, a slit sampler, an Andersen sampler, a proton impinger, an electrostatic precipitator, and an impinger such as a capillary impinger or a liquid impinger. In some preferred embodiments, the sampler is modified by inserting a chip containing an LC film inside the collection chamber of the sampler. In some preferred embodiments, the LC film is provided as a liquid crystal detection unit as described in more detail herein. In some embodiments, the liquid crystal detection unit comprises an adhesive backing allowing attachment to the collection chamber.

In other preferred embodiments, the liquid crystal film is placed in an environment occupied by people, air from the environment is sampled, and an atomizer generates small water droplets that is mixed with the air from the environment and applied on the surface of a liquid crystal detection unit. The presence of an entity comprising a biological membrane may be detected as described above.

The scope of the invention includes a wide range of methods that can be used to create liquid crystal-air interfaces, including the use of microwells, but also including droplets of liquid crystal presented at a surface, and films of liquid crystals that are stabilized by polymer networks, colloidal networks or other gel-forming internal structures as is known in the art.

In some preferred embodiments, artificial intelligence algorithms are used in conjunction with the LC analysis methods and devices described below to determine if an LC film has been exposed to a biological entity comprising a lipid membrane. In some preferred embodiments, the algorithms provide an analysis of whether the orientation of mesogens in the LC has been changed due to the presence of a biological entity comprising a lipid membrane in a aqueous aerosol. In some embodiments, an image of the LC film is captured and transmitted to a central server or site. An artificial intelligence algorithm is then utilized to analyze the image and return to the user and determination of whether a biological entity comprising a lipid membrane, such as a virus, has been detected. The user may then be directed to proceed to have another tests performed for the biological entity, such as an antibody-based test (e.g., an ELISA) or a nucleic acid-based test (e.g., a PCR assay).

### I. Breathing Barriers

In some preferred embodiments, the present invention provides breathing barriers into which an LC detection unit has been incorporated. The present invention is not limited to the use of any particular type of breathing barrier. Indeed, a variety of breathing barriers are useful in the present invention including, but not limited to, face masks, respirators, surgical masks, and face shields.

Face masks include but are not limited to cloth face coverings and surgical masks.

In general, the CDC recommends that members of the public use simple cloth face coverings (such as clot face masks) when in a public setting to slow the spread of viruses, since this will help people who may have the virus and do not know it from transmitting it to others. In some preferred embodiments, it is contemplated that LC detectors can be incorporated into cloth face covering. In some embodiments, it is contemplated that the LC detectors have an adhesive backing and can be attached to the skin of a person in an area that lies beneath a cloth face covering or face mask. In some embodiments, it is contemplated that LC detectors can be incorporated inside device used for collecting aerosols (a “bioaerosol sampler”) and used for monitoring worker exposure in the context of industrial hygiene.

A surgical mask is a loose-fitting, disposable device that creates a physical barrier between the mouth and nose of the wearer and potential contaminants in the immediate environment. Surgical masks are regulated under 21 CFR 878.4040. Surgical masks are not to be shared and may be labeled as surgical, isolation, dental, or medical procedure masks. In some preferred embodiments, the present invention contemplates incorporation of LC detection devices into these different types of surgical masks. Surgical masks may come with or without a face shield. Surgical masks are often referred to as face masks, although not all face masks are regulated as surgical masks. Surgical masks are made in different thicknesses and with different ability to protect the user from contact with liquids.

In some preferred embodiments, it is contemplated that the LC detection units are incorporated into a respirator. A respirator is a device designed to protect the wearer from inhaling hazardous atmospheres, including fumes, vapors, gases and particulate matter such as dusts and airborne microorganisms. There are two main categories: the air-purifying respirator, in which respirable air is obtained by filtering a contaminated atmosphere, and the air-supplied respirator, in which an alternate supply of breathable air is delivered. Within each category, different techniques are employed to reduce or eliminate noxious airborne contaminants. The LC detection units of the present invention may be incorporated into any type of respirator. In some particularly preferred embodiments, the respirator is an air purifying respirator. Air-purifying respirators include, but are not limited to, single-use, disposable face masks (commonly referred to as a dust mask) to reusable models with replaceable cartridges (commonly referred to a gas mask).

In some preferred embodiments, the respirator is an N95 or KN95 respirator. An N95 respirator is a respiratory protective device designed to achieve a very close facial fit and very efficient filtration of airborne particles. The ‘N95’ designation means that when subjected to careful testing, the respirator blocks at least 95 percent of very small (0.3 micron) test particles. If properly fitted, the filtration capabilities of N95 respirators exceed those of face masks.

Most N95 respirators are manufactured for use in construction and other industrial type jobs that expose workers to dust and small particles. They are regulated by the National Personal Protective Technology Laboratory (NPPTL) in the National Institute for Occupational Safety and Health (NIOSH), which is part of the Centers for Disease Control and Prevention (CDC). However, some N95 respirators are intended for use in a health care setting. Specifically, single-use, disposable respiratory protective devices used and worn by health care personnel during procedures to protect both the patient and health care personnel from the transfer of microorganisms, body fluids, and particulate material. These surgical N95 respirators are class II devices regulated by the FDA, under 21 CFR 878.4040, and CDC NIOSH under 42 CFR Part 84.

In some preferred embodiments, it is contemplated that the LC detection units are incorporated into a face shield. A face shield protects the wearer's entire face (or part of it) from hazards such as flying objects and road debris, chemical splashes (in laboratories or in industry), or potentially infectious materials (in medical and laboratory environments). In some preferred embodiments, face shields are formed from a polymeric material.

### II. LC Detection Units

As discussed above, the present invention utilizes an LC detection unit in combination with the breathing barrier. Suitable LC detection units and methodologies are described in U.S. Pat. Nos. 8,988,620 and 9,816,147, each of which is incorporated herein by reference in its entirety.

Substrates that are useful in practicing the present invention can be made of practically any physicochemically stable material. In a preferred embodiment, the substrate material is non-reactive towards the constituents of the mesogenic layer. The substrates can be either rigid or flexible and can be either optically transparent or optically opaque. The substrates can be electrical insulators, conductors or semiconductors. Further, the substrates can be substantially impermeable to liquids, vapors and/or gases or, alternatively, the substrates can be permeable to one or more of these classes of materials. Exemplary substrate materials include, but are not limited to, inorganic crystals, inorganic glasses, inorganic oxides, metals, organic polymers and combinations thereof. In some embodiments, the substrates have microchannels therein for the delivery of sample and/or other reagents to the substrate surface or detection regions thereon. The design and use of microchannels are described, for example, in U.S. Pat. Nos. 6,425,972, 6,418,968, 6,447,727, 6,432,720, 5,976,336, 5,882,465, 5,876,675, 6,186,660, 6,100,541, 6,379,974, 6,267,858, 6,251,343, 6,238,538, 6,182,733, 6,068,752, 6,429,025, 6,413,782, 6,274,089, 6,150,180, 6,046,056, 6,358,387, 6,321,791, 6,326,083, 6,171,067, and 6,167,910, all of which are incorporated herein by reference.

In some embodiments of the present invention, inorganic crystals and inorganic glasses are utilized as substrate materials (e.g., LiF, NaF, NaCl, KBr, KI, CaF2, MgF2, HgF2, BN, AsS3, ZnS, Si3N4 and the like). The crystals and glasses can be prepared by art standard techniques (See, e.g., Goodman, C. H. L., Crystal Growth Theory and Techniques, Plenum Press, New York 1974). Alternatively, the crystals can be purchased commercially (e.g., Fischer Scientific). The crystals can be the sole component of the substrate or they can be coated with one or more additional substrate components. Thus, it is within the scope of the present invention to utilize crystals coated with, for example one or more metal films or a metal film and an organic polymer. Additionally, a crystal can constitute a portion of a substrate which contacts another portion of the substrate made of a different material, or a different physical form (e.g., a glass) of the same material. Other useful substrate configurations utilizing inorganic crystals and/or glasses will be apparent to those of skill in the art.

In other embodiments of the present invention, inorganic oxides are utilized as the substrate. Inorganic oxides of use in the present invention include, for example, Cs20, Mg(OH)2, Ti02, Zr02, Ce02, Y203, Cr203, Fe203, NiO, ZnO, Al203, Si02 (glass), quartz, In203, Sn02, Pb02 and the like. The inorganic oxides can be utilized in a variety of physical forms such as films, supported powders, glasses, crystals and the like. A substrate can consist of a single inorganic oxide or a composite of more than one inorganic oxide. For example, a composite of inorganic oxides can have a layered structure (i.e., a second oxide deposited on a first oxide) or two or more oxides can be arranged in a contiguous non layered structure. In addition, one or more oxides can be admixed as particles of various sizes and deposited on a support such as a glass or metal sheet. Further, a layer of one or more inorganic oxides can be intercalated between two other substrate layers (e.g., metal oxide metal, metal oxide-crystal).

In a presently preferred embodiment, the substrate is a rigid structure that is impermeable to liquids and gases. In this embodiment, the substrate consists of a glass plate onto which a metal, such as gold is layered by evaporative deposition. In a still further preferred embodiment, the substrate is a glass plate (Si02) onto which a first metal layer such as titanium has been layered. A layer of a second metal such as gold is then layered on top of the first metal layer.

In still further embodiments of the present invention, metals are utilized as substrates. The metal can be used as a crystal, a sheet or a powder. The metal can be deposited onto a backing by any method known to those of skill in the art including, but not limited to, evaporative deposition, sputtering, electroless deposition, electrolytic deposition and adsorption or deposition of preform particles of the metal including metallic nanoparticles.

Any metal that is chemically inert towards the mesogenic layer will be useful as a substrate in the present invention. Metals that are reactive or interactive towards the mesogenic layer will also be useful in the present invention. Metals that are presently preferred as substrates include, but are not limited to, gold, silver, platinum, palladium, nickel and copper. In one embodiment, more than one metal is used. The more than one metal can be present as an alloy or they can be formed into a layered “sandwich” structure, or they can be laterally adjacent to one another. In a preferred embodiment, the metal used for the substrate is gold. In a particularly preferred embodiment the metal used is gold layered on titanium.

The metal layers can be either permeable or impermeable to materials such as liquids, solutions, vapors and gases.

In still other embodiments of the present invention, organic polymers are utilized as substrate materials. Organic polymers useful as substrates in the present invention include polymers that are permeable to gases, liquids and molecules in solution. Other useful polymers are those that are impermeable to one or more of these same classes of compounds.

Organic polymers that form useful substrates include, for example, polyalkenes (e.g., polyethylene, polyisobutene, polybutadiene), polyacrylics (e.g., polyacrylate, polymethyl methacrylate, polycyanoacrylate), polyvinyls (e.g., polyvinyl alcohol, polyvinyl acetate, polyvinyl butyral, polyvinyl chloride), polystyrenes, polycarbonates, polyesters, polyurethanes, polyamides, polyimides, polysulfone, polysiloxanes, polyheterocycles, cellulose derivative (e.g., methyl cellulose, cellulose acetate, nitrocellulose), polysilanes, fluorinated polymers, epoxies, polyethers and phenolic resins (See, Cognard, J. ALIGNMENT OF NEMATIC LIQUID CRYSTALS AND THEIR MIXTURES, in Mol. Cryst. Liq. Cryst. 1:1 74 (1982)). Presently preferred organic polymers include polydimethylsiloxane, polyethylene, polyacrylonitrile, cellulosic materials, polycarbonates and polyvinyl pyridinium.

In some embodiments, the surface of the substrate is functionalized. In some embodiments, the surface of the substrate is first functionalized by forming a self-assembled monolayer (SAM) on the substrate surface.

Self assembled monolayers are generally depicted as an assembly of organized, closely packed linear molecules. There are two widely used methods to deposit molecular monolayers on solid substrates: Langmuir Blodgett transfer and self assembly. Additional methods include techniques such as depositing a vapor of the monolayer precursor onto a substrate surface and the layer-by-layer deposition of polymers and polyelectrolytes from solution (Ladam et al., Protein Adsorption onto Auto-Assembled Polyelectrolyte Films, Langmuir; 2001; 17(3); 878-882).

The composition of a layer of a SAM useful in the present invention can be varied over a wide range of compound structures and molar ratios. In one embodiment, the SAM is formed from only one compound. In a presently preferred embodiment, the SAM is formed from two or more components. In another preferred embodiment, when two or more components are used, one component is a long chain hydrocarbon having a chain length of between 10 and 25 carbons and a second component is a short chain hydrocarbon having a chain length of between 1 and 9 carbon atoms. In particularly preferred embodiments, the SAM is formed from CH₃(CH₂)₁₅SH and CH₃(CH₂)₄SH or CH₃(CH₂)₁₅SH and CH₃(CH₂)₉SH. In any of the above described embodiments, the carbon chains can be functionalized at the terminus (e.g., NH₂, COOH, OH, CN), at internal positions of the chain (e.g., aza, oxa, thia) or at both a terminus and internal positions of the chain.

In some embodiments, the SAM may be functionalized. Currently favored classes of reactions available with reactive SAM components are those that proceed under relatively mild conditions. These include, but are not limited to nucleophilic substitutions (e.g., reactions of amines and alcohols with acyl halides), electrophilic substitutions (e.g., enamine reactions) and additions to carbon and carbon heteroatom multiple bonds (e.g., Michael reaction, Diels Alder addition). These and other useful reactions are discussed in March, ADVANCED ORGANIC CHEMISTRY, Third Ed., John Wiley & Sons, New York, 1985.

According to the present invention, a substrate's surface is functionalized with SAM, components and other species by covalently binding a reactive SAM component to the substrate surface in such a way as to derivatize the substrate surface with a plurality of available reactive functional groups. Reactive groups which can be used in practicing the present invention include, for example, amines, hydroxyl groups, carboxylic acids, carboxylic acid derivatives, alkenes, sulfhydryls, siloxanes, etc.

A wide variety of reaction types are available for the functionalization of a substrate surface. For example, substrates constructed of a plastic such as polypropylene, can be surface derivatized by chromic acid oxidation, and subsequently converted to hydroxylated or aminomethylated surfaces. Substrates made from highly crosslinked divinylbenzene can be surface derivatized by chloromethylation and subsequent functional group manipulation. Additionally, functionalized substrates can be made from etched, reduced polytetrafluoroethylene.

When the substrates are constructed of a siliaceous material such as glass, the surface can be derivatized by reacting the surface Si OH, Si0 H, and/or Si groups with a functionalizing reagent. When the substrate is made of a metal film, the surface can be derivatized with a material displaying avidity for that metal.

It will be apparent to those of skill in the art that an array of similarly useful functionalizing chemistries are available when SAM components other than siloxanes are used. Thus, for example similarly functionalized alkyl thiols can be attached to metal films and subsequently reacted to produce the functional groups.

In another preferred embodiment, the substrate is at least partially a metal film, such as a gold film, and the reactive group is tethered to the metal surface by an agent displaying avidity for that surface.

In some embodiments, the substrates are coated with polyimide layer. It is contemplated that polyimide coated substrates are especially useful because in some instances, the surfaces homeotropically orient a liquid crystal, while in other instances the surfaces can be rubbed to provide an anisotropic surface for orient a liquid crystal. In preferred embodiments, a substrate such as a silicon wafer is coated with a polyimide. In preferred embodiment, the substrate is spin coated with the polyimide. A variety of polyimides find use with the present invention, including, but not limited to Nissan 7210, Nissan 3510, Nissan 410, Nissan 3140, Nissan 5291, and Japan Synthetic Rubber JALS 146-R19 for planar alignment of liquid crystals and Nissan 7511L and SE 1211 for homeotropic orientation of liquid crystals.

Any compound or mixture of compounds which forms a mesogenic layer can be used in conjunction with the present invention. The mesogens can form thermotropic or lyotropic liquid crystals. Both the thermotropic and lyotropic liquid crystals can exist in a number of forms including nematic, chiral nematic, smectic, polar smectic, chiral smectic, frustrated phases and discotic phases.

Presently preferred mesogens are displayed in Table 1. In a particularly preferred embodiment, the mesogen is a member selected from the group consisting of 4 cyano 4′ pentylbiphenyl, N (4methoxybenzylidene) 4 butlyaniline and combinations thereof. The mesogenic layer can be a substantially pure compound, or it can contain other compounds which enhance or alter characteristics of the mesogen. Thus, in one preferred embodiment, the mesogenic layer further comprises a second compound, for example and alkane, which expands the temperature range over which the nematic and isotropic phases exist. Use of devices having mesogenic layers of this composition allows for detection of the analyte recognition moiety interaction over a greater temperature range.

TABLE 1
Molecular structure of mesogens suitable for use in Liquid
Crystal Assay Devices

| Mesogen | Structure |
|---|---|
| Anisaldazine | 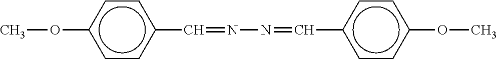 |
| NCB | 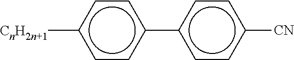 |
| CBOOA | 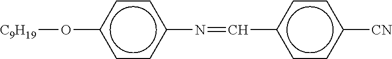 |
| Comp A | 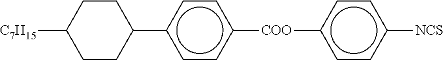 |
| Comp B | 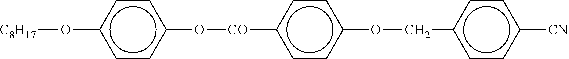 |
| DB₇NO₂ | 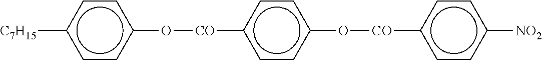 |
| DOBAMBC | 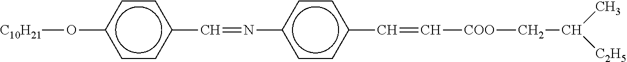 |
| nOm n = 1, m = 4: MBBA n = 2, m = 4: EBBA | 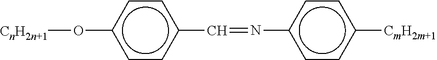 |
| nOBA n = 8: OOBA n = 9: NOBA | 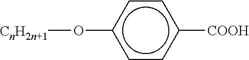 |
| nmOBC | 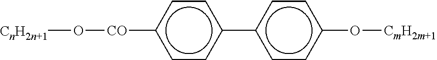 |
| nOCB | 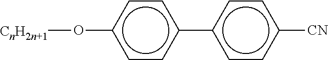 |
| nOSI | 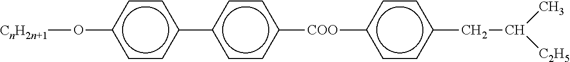 |
| 98P | 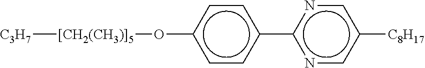 |
| PAA | 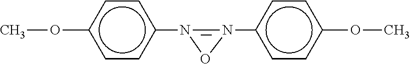 |
| PYP906 | 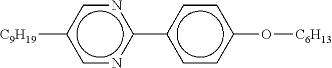 |
| n̅Sm | 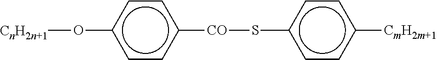 |

In some preferred embodiments, the mesogenic layer further comprises a dichroic dye or fluorescent compound. Examples of dichroic dyes and fluorescent compounds useful in the present invention include, but are not limited to, azobenzene, BTBP, polyazo compounds, anthraquinone, perylene dyes, and the like. In particularly preferred embodiments, a dichroic dye of fluorescent compound is selected that complements the orientation dependence of the liquid crystal so that polarized light is not required to read the assay. In some preferred embodiments, if the absorbance of the liquid crystal is in the visible range, then changes in orientation can be observed using ambient light without crossed polars. In other preferred embodiments, the dichroic dye or fluorescent compound is used in combination with a fluorimeter and the changes in fluorescence are used to detect changes in orientation of the liquid crystal.

In some preferred embodiments, the LC detection devices comprise one or more recognition moieties. In some embodiments, the breath of the individual creates an LC/aqueous interface in the LC detection device and the recognition moieties are provided at the LC/aqueous interface. In some embodiments, the recognition moiety may be positioned at the LC/aqueous interface by modifying the recognition moiety to include a lipid tail. A variety of recognition moieties find use in the present invention.

In some preferred embodiments, the recognition moiety is a biomolecule such as a protein, nucleic acid, peptide or an antibody. Biomolecules useful in practicing the present invention can be derived from any source. The biomolecules can be isolated from natural sources or can be produced by synthetic methods. Proteins can be natural proteins or mutated proteins. Mutations can be effected by chemical mutagenesis, site-directed mutagenesis or other means of inducing mutations known to those of skill in the art. Proteins useful in practicing the instant invention include, for example, enzymes, antigens, antibodies and receptors. Antibodies can be either polyclonal or monoclonal. Peptides and nucleic acids can be isolated from natural sources or can be wholly or partially synthetic in origin. Examples of antigen binding proteins finding use in the present invention include, but are not limited to, immunoglobulins, single chain antibodies, chimeric antibodies, polyclonal antibodies, monoclonal antibodies, and F(ab′)₂, Fab′ and Fab fragments.

Various procedures known in the art may be used for the production of polyclonal antibodies. For the production of antibody, various host animals, including but not limited to rabbits, mice, rats, sheep, goats, etc., can be immunized by injection with the peptide corresponding to an epitope. In a preferred embodiment, the peptide is conjugated to an immunogenic carrier (e.g., diphtheria toxoid, bovine serum albumin (BSA), or keyhole limpet hemocyanin (KLH)). Various adjuvants may be used to increase the immunological response, depending on the host species, including but not limited to Freund's (complete and incomplete), mineral gels (e.g., aluminum hydroxide), surface active substances (e.g., lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum).

For preparation of monoclonal antibodies, it is contemplated that any technique that provides for the production of antibody molecules by continuous cell lines in culture will find use with the present invention (See e.g., Harlow and Lane, Antibodies: A Laboratory Manual, Cold Spring Harbor Laboratory Press, Cold Spring Harbor, N.Y.). These include but are not limited to the hybridoma technique originally developed by Kohler and Milstein (Kohler and Milstein, Nature 256:495-497 [1975]), as well as the trioma technique, the human B-cell hybridoma technique (See e.g., Kozbor et al., Immunol. Tod., 4:72 [1983]), and the EBV-hybridoma technique to produce human monoclonal antibodies (Cole et al., in Monoclonal Antibodies and Cancer Therapy, Alan R. Liss, Inc., pp. 77-96 [1985]).

In addition, it is contemplated that techniques described for the production of single chain antibodies (U.S. Pat. No. 4,946,778; herein incorporated by reference) will find use in producing specific single chain antibodies that serve as recognition moieties. Furthermore, it is contemplated that any technique suitable for producing antibody fragments will find use in generating antibody fragments that are useful recognition moieties. For example, such fragments include but are not limited to: F(ab′)2 fragment that can be produced by pepsin digestion of the antibody molecule; Fab′ fragments that can be generated by reducing the disulfide bridges of the F(ab′)2 fragment, and Fab fragments that can be generated by treating the antibody molecule with papain and a reducing agent. In still further embodiments, the recognition moiety comprises a phage displaying an antigen binding protein.

In other embodiments, the recognition moieties can be nucleic acids (e.g., RNA or DNA) or receptors that are specific for a particular entity (e.g., virus). In some embodiments, the nucleic acids are aptamers. The isolation of aptamers is described in U.S. Pat. Nos. 5,475,096; 5,270,163; and 5,475,096; and in PCT publications WO 97/38134, WO 98/33941, and WO 99/07724, all of which are herein incorporated by reference.

In some embodiments, recognition moieties are incorporated to detect a variety of bacteria and pathogens. Such recognition moieties include, but not limited to, sialic acid to detect HIV (Wies et al., Nature 333: 426 [1988]), influenza (White et al., Cell 56: 725 [1989]), chlamydia (Infect. Imm. 57: 2378 [1989]), reovirus, Streptococcus suis, Salmonella, Sendai virus, mumps, newcastle, myxovirus, and Neisseria meningitidis; 9-OAC sialic acid to detect coronavirus, encephalomyelitis virus, and rotavirus; non-sialic acid glycoproteins to detect cytomegalovirus (Virology 176: 337 [1990]) and measles virus (Virology 172: 386 [1989]); CD4 (Khatzman et al., Nature 312: 763 [1985]), vasoactive intestinal peptide (Sacerdote et al., J. of Neuroscience Research 18: 102 [1987]), and peptide T (Ruff et al., FEBS Letters 211: 17 [1987]) to detect HIV; epidermal growth factor to detect vaccinia (Epstein et al., Nature 318: 663 [1985]); acetylcholine receptor to detect rabies (Lentz et al., Science 215: 182 [1982]); Cd3 complement receptor to detect Epstein-Barr virus (Carel et al., J. Biol. Chem. 265: 12293 [1990]); β-adrenergic receptor to detect rheovirus (Co et al., Proc. Natl. Acad. Sci. 82: 1494 [1985]); ICAM-1 (Marlin et al., Nature 344: 70 [1990]), N-CAM, and myelin-associated glycoprotein MAb (Shephey et al., Proc. Natl. Acad. Sci. 85: 7743 [1988]) to detect rhinovirus; polio virus receptor to detect polio virus (Mendelsohn et al., Cell 56: 855 [1989]); fibroblast growth factor receptor to detect herpesvirus (Kaner et al., Science 248: 1410 [1990]); oligomannose to detect Escherichia coli; ganglioside G_{M}1 to detect Neisseria meningitidis; and antibodies to detect a broad variety of pathogens (e.g., Neisseria gonorrhoeae, V. vulnificus, V. parahaemolyticus, V. cholerae, V. alginolyticus, etc.).

In still further embodiments, the recognition moiety is a ligand that interacts with a binding partner. Examples of ligands include, but are not limited to, small organic molecules such as steroid molecules and small drug molecules, proteins, polypeptides and peptides, metal ions, and nucleic acids. In some embodiments, the ligand is recognized by a binding molecule in a sample. Examples of binding molecules include, but are not limited to, steroids, hormones, proteins, polypeptides, and peptides such immunoglobulin molecules and fragments thereof, nucleic acids, and other organic or non-organic molecules. In some preferred embodiments, the ligand is recognized by a binding molecule in a body fluid of a test subject. For example, the ligand can be a virus envelope protein or some other antigenic molecule from a pathogenic organism (such as those listed above). In preferred embodiments, the antigenic molecule (e.g., a protein) is recognized by an antibody molecule in the body fluid of a test subject that has been exposed to the pathogenic organism. In particularly preferred embodiments, the ligand is protein E from the envelope of West Nile Virus.

In some preferred embodiments, the ligands or recognition moieties are complexed with a lipid. The present invention contemplates complexation of the recognition moiety with a variety of lipids and lipid containing materials, including, but not limited to, fatty acids, phospholipids, mono-, di- and tri-glycerides comprising fatty acids and/or phospholipids, lipid bilayers, and liposomes. The lipid containing material can be provided as multilayers, as well as braided, lamellar, helical, tubular, and fiber-like shapes, and combinations thereof. Standard attachment chemistries are available for attaching a recognition moiety or ligand of interest to lipids and lipids containing materials. These attachment chemistries are described in more detail below with reference to liposomes.

III. Detection of Entities with Lipid Membranes

The present invention provides methods and devices for the direct detection of entities having a biological membrane, including viruses and bacteria that are pathogens, in the respiratory droplets of a subject. The systems and devices of the present invention can be of any configuration that allows for the contact of a mesogenic layer with the breath of a subject. The only limitations on size and shape are those that arise from the situation in which the device is used or the purpose for which it is intended. The device can be planar or non-planar. Thus, it is within the scope of the present invention to use any number of polarizers, lenses, filters lights, and the like to practice the present invention.

The systems and devices of the present invention find use in the detection of variety of viruses and entities having lipid membranes. Examples of such entities having lipid membranes include, but are not limited to, viruses, bacteria, liposomes, cells, mycoplasmas, protozoans, fungi and the like.

The present invention is not limited to the detection of any particular type of virus. Indeed, the present invention contemplates the detection of a variety of viruses, including viruses from the following families: Adenoviridae, Arenaviridae, Astroviridae, Birnaviridae, Bunyaviridae, Caliciviridae, Circoviridae, Coronaviridae, Filoviridae, Flaviviridae, Hepadnaviridae, Herpesviridae, Iridoviridae, Filoviridae, Orthomyxoviridae, Papovaviridae, Paramyxoviridae, Parvoviridae, Picornaviridae, Poxviridae, Reoviridae, Retroviridae, Rhabdoviridae, Togaviridae, Badnavirus, Bromoviridae, Comoviridae, Geminiviridae, Partitiviridae, Potyviridae, Sequiviridae, and Tombusviridae; the following genera: Mastadenovirus, Aviadenovirus, African swine fever-like viruses, Arenavirus, Arterivirus, Astrovirus, Aquabirnavirus, Avibirnavirus, Bunyavirus, Hantavirus, Nairovirus, Phlebovirus, Calicivirus, Circovirus, Coronavirus, Torovirus, Deltavirus, Filovirus, Flavivirus, Japanese Encephalitis Virus group, Pestivirus, Hepatitis C-like viruses, Orthohepadnavirus, Avihepadnavirus, Simplexvirus, Varicellovirus, Cytomegalovirus, Muromegalovirus, Roseolovirus, Lymphocryptovirus, Rhadinovirus, Ranavirus, Lymphocystivirus, Goldfish virus-like viruses, Influenzavirus A, B, Influenzavirus C, Thogoto-Like viruses, Polyomavirus, Papillomavirus, Paramyxovirus, Morbillivirus, Rubulavirus, Pneumovirus, Parvovirus, Erythrovirus, Dependovirus, Enterovirus, Rhinovirus, Hepatovirus, Cardiovirus, Aphthovirus, Orthopoxvirus, Parapoxvirus, Avipoxvirus, Capripoxvirus, Leporipoxvirus, Suipoxvirus, Molluscipoxvirus, Yatapoxvirus, Orthoreovirus, Orbivirus, Rotavirus, Coltivirus, Aquareovirus, mammalian type B retroviruses, mammalian type C retroviruses, avian type C retroviruses, type D retroviruses, blv-htiv retroviruses, Lentivirus, Spumavirus, Vesiculovirus, Lyssavirus, Ephemerovirus, Alphavirus, Rubivirus, Badnavirus, Alfamovirus, Ilarvirus, Bromovirus, Cucumovirus, Tospovirus, Capillovirus, Carlavirus, Caulimovirus, Closterovirus, Comovirus, Fabavirus, Nepovirus, Dianthovirus, Enamovirus, Furovirus, Subgroup I Geminivirus, Subgroup II Geminivirus, Subgroup III Geminivirus, Hordeivirus, Idaeovirus, Luteovirus, Machlomovirus, Marafivirus, Necrovirus, Partitiviridae, Alphacryptovirus, Betacryptovirus, Potexvirus, Potyvirus, Rymovirus, Bymovirus, Fijivirus, Phytoreovirus, Oryzavirus, Nucleorhabdovirus, Sequivirus, Waikavirus, Sobemovirus, Tenuivirus, Tobamovirus, Tobravirus, Carmovirus, Tombusvirus, Trichovirus, Tymovirus, Umbravirus; and the following species: human adenovirus 2, fowl adenovirus 1, African swine fever virus, lymphocytic choriomeningitis virus, equine arteritis virus, human astrovirus 1, infectious pancreatic necrosis virus, infectious bursal disease virus, Bunyamwera virus, Hantaan virus, Nairobi sheep disease virus, sandfly fever Sicilian virus, vesicular exanthema of swine virus, chicken anemia virus, avian infectious bronchitis virus, Berne virus, hepatitis delta virus, Marburg virus, yellow fever virus, west Nile virus, bovine diarrhea virus, hepatitis C virus, hepatitis B virus, duck hepatitis B virus, human herpesvirus 1, human herpesvirus 3, human herpesvirus 5, human cytomegalovirus, mouse cytomegalovirus 1, human herpesvirus 6, human herpesvirus 4, ateline herpesvirus 2, frog virus 3, flounder virus, goldfish virus 1, influenza A virus, influenza B virus, influenza C virus, Thogoto virus, murine polyomavirus, cottontail rabbit papillomavirus (Shope), Paramyxovirus, human parainfluenza virus 1, measles virus, mumps virus, human respiratory syncytial virus, mice minute virus, B19 virus, adeno-associated virus 2, poliovirus 1, human rhinovirus 1A, porcine rhinovirus, hepatitis A virus, encephalomyocarditis virus, St. Louis encephalomyocarditis virus, foot-and-mouth disease virus 0, vaccinia virus, orf virus, fowlpox virus, sheeppox virus, monkey pox virus, myxoma virus, swinepox virus, Molluscum contagiosum virus, Yaba monkey tumor virus, reovirus 3, bluetongue virus 1, simian rotavirus SA11, Colorado tick fever virus, golden shiner virus, mouse mammary tumor virus, murine leukemia virus, avian leukosis virus, Mason-Pfizer monkey virus, bovine leukemia virus, human immunodeficiency virus 1, human spumavirus, vesicular stomatitis Indiana virus, rabies virus, bovine ephemeral fever virus, Sindbis virus, and rubella virus.

In some preferred embodiments, the virus is an enveloped virus. In some particularly preferred embodiments, the virus is a corona virus such as SARS-CoV-1, SARS-CoV-2, MERS or an influenza virus.

The present invention is not limited to the detection of any particular type of bacteria. Indeed, the detection of variety of bacteria is contemplated, including, but not limited to Gram-positive cocci such as Staphylococcus aureus, Streptococcus pyogenes (group A), Streptococcus spp. (viridans group), Streptococcus agalactiae (group B), S. bovis, Streptococcus (anaerobic species), Streptococcus pneumoniae, and Enterococcus spp.; Gram-negative cocci such as Neisseria gonorrhoeae, Neisseria meningitidis, and Branhamella catarrhalis; Gram-positive bacilli such as Bacillus anthracis, Bacillus subtilis, Corynebacterium diphtheriae and Corynebacterium species which are diptheroids (aerobic and anerobic), Listeria monocytogenes, Clostridium tetani, Clostridium difficile, Escherichia coli, Enterobacter species, Proteus mirablis and other spp., Pseudomonas aeruginosa, Klebsiella pneumoniae, Campylobacter jejuni, Legionella peomophilia, Mycobacterium tuberculosis, Clostridium tetani, Hemophilus influenzae, Neisseria gonorrhoeae, Treponema pallidum, Bacillus anthracis, Vibrio cholerae, Borrelia burgdorferi, Cornebacterium diphtheria, Staphylococcus aureus, Bacillus anthracis, and other members of the following genera: Vibrio, Salmonella, Shigella, Pseudomonas, Actinomyces, Aeromonas, Bacillus, Bacteroides, Bordetella, Brucella, Campylobacter, Capnbocylophaga, Clamydia, Clostridium, Corynebacterium, Eikenella, Erysipelothriz, Escherichia, Fusobacterium, Hemophilus, Klebsiella, Legionella, Leptospira, Listeria, Mycobacterium, Mycoplasma, Neisseria, Nocardia, Pasteurella, Proteus, Pseudomonas, Rickettsia, Salmonella, Selenomonas, Shigella, Staphylococcus, Streptococcus, Treponema, Bibro, and Yersinia. Bacterial infections result in diseases such as bacteremia, pneumonia, meningitis, osteomyelitis, endocarditis, sinusitis, arthritis, urinary tract infections, tetanus, gangrene, colitis, acute gastroenteritis, bronchitis, and a variety of abscesses, nosocomial infections, and opportunistic infections.

As discussed above, in some preferred embodiments, the presence of a biological entity comprising a lipid membrane in respiratory droplets may be detected by changes associated with the mesogens in LC layer of the LC detection unit.

The present invention is not limited to any particular method of detection a change in the orientation of the mesogens in the device. Thus, it is within the scope of the present invention to use lights, microscopes, spectrometry, electrical techniques and the like to aid in the detection of a change in the mesogenic layer. In those embodiments utilizing light in the visible region of the spectrum, the light can be used to simply illuminate details of the mesogenic layer to provide for visual detection. Alternatively, the light can be passed through the mesogenic layer and the amount of light transmitted, absorbed or reflected can be measured. The device can utilize a backlighting device such as that described in U.S. Pat. No. 5,739,879. Light in the ultraviolet and infrared regions is also of use in the present invention. Microscopic techniques can utilize simple light microscopy, confocal microscopy, polarized light microscopy, atomic force microscopy (Hu et al., Langmuir 13:5114-5119 (1997)), scanning tunneling microscopy (Evoy et al., J. Vac. Sci. Technol A 15:1438-1441, Part 2 (1997)), and the like. Spectroscopic techniques of use in practicing the present invention include, for example, infrared spectroscopy (Zhao et al., Langmuir 13:2359-2362 (1997)), raman spectroscopy (Zhu et al., Chem. Phys. Lett. 265:334-340 (1997)), X-ray photoelectron spectroscopy (Jiang et al., Bioelectroch. Bioener. 42:15-23 (1997)) and the like. Visible and ultraviolet spectroscopies are also of use in the present invention. Other useful techniques include, for example, surface plasmon resonance (Evans et al., J. Phys. Chem. B 101:2143-2148 (1997), ellipsometry (Harke et al., Thin Solid Films 285:412-416 (1996)), electrical methods (such as impedometric methods (Rickert et al., Biosens. Bioelectron. 11:757:768 (1996)), and the like.

In some embodiments, the devices of the present invention further comprise an electrode or series of electrodes. In some preferred embodiments, at least two electrodes are provided in a plane on one of the surfaces of the device substrate. A variety of electrodes may be utilized, including, but not limited to, interdigitated, hyperbolic, triangular and rectangular electrodes. In some particularly preferred embodiments, the device comprises interdigitated electrodes. In preferred embodiments, the electrodes are utilized to transfer viral or other particles to a surface of the assay device, preferably to a surface comprising recognition moieties.

The electrodes are also utilized to measure changes in dielectric capacitance of the device that is associated with a change in mesogens due to the presence of an entity comprising a lipid membrane. The present invention is not limited to a particular mechanism of action. Indeed, an understanding of the mechanism of action is not necessary to practice the present invention. Nevertheless, it is contemplated that liquid crystals have large, anisotropic electrical properties that are reflected in changes in electrical capacitance related to orientation within an electrical field. The method of the present invention, based on dielectric transduction, relies on the principle of change in capacitance between two electrodes when dielectric properties of the medium between them changes.

In some preferred embodiments, the breath of subjects suspected of containing a virus or entity having a lipid membrane or in need of monitoring is allowed to contact the LC in the LC detection device for a pre-determined amount of time. Following contact with the LC, the cell is assayed for whether a change in the LC has occurred. Although many changes in the mesogenic layer can be detected by visual observation under ambient light, any means for detecting the change in the mesogenic layer can be incorporated into, or used in conjunction with, the device. Thus, it is within the scope of the present invention to use lights, microscopes, spectrometry, electrical techniques and the like to aid in the detection of a change in the mesogenic layer. In some embodiments, binding of virus to the virus recognition moiety is detected by a change in the color and texture of the liquid crystal. The present invention is not limited to any particular mechanism of action. Indeed, an understanding of the mechanism of action is not necessary to practice the invention. Nevertheless, it is believed that the change in color and texture is due tilting of the mesogens in the liquid crystal prior to assumption of a homeotropic orientation.

Accordingly, in those embodiments utilizing light in the visible region of the spectrum, the light can be used to simply illuminate details of the mesogenic layer. Alternatively, the light can be passed through the mesogenic layer and the amount of light transmitted, absorbed or reflected can be measured. The device can utilize a backlighting device such as that described in U.S. Pat. No. 5,739,879, incorporated herein by reference. Light in the ultraviolet and infrared regions is also of use in the present invention.

In some embodiments, the cell in the LC detection unit is placed in between cross polar lenses and light is passed though the lenses and the cell. Areas of homeotropic orientation appear black, while areas of planar orientation appear bright. Thus, the presence of bound virus is indicated by a black field while areas where no virus is bound are indicated by a bright field.

In some embodiments, the present invention utilizes plate readers to detect changes in the orientation of mesogens in the LC detection unit. In particular, the present invention includes methods and processes for the quantification of light transmission through films of liquid crystals based on quantification of transmitted or reflected light.

The present invention is not limited to any particular mechanism of action. Indeed, an understanding of the mechanism of action is not required to practice the present invention. Nevertheless, it is contemplated that ordered nanostructured substrates impart order to thin films of liquid crystal placed onto their surface. These ordered films of liquid crystal preserve the plane of polarized light passed through them. If the liquid crystal possesses a well-defined distortion—such as a 90 degree twist distortion—then the liquid crystal will change the polarization of the transmitted light in a well-defined and predictable manner. It is further contemplated that ordered films (e.g., areas of homeotropic orientation) of liquid crystal differentially absorb (relative to randomly ordered films of liquid crystal) specific wavelengths of light.

Accordingly, the present invention contemplates the use of plate readers to detect light transmission through an LC detection unit when viewed through cross polars, the transmission of light through an LC detection unit illuminated with a suitable wavelength of light, or reflection of light (i.e., polarized light or non-polarized light of specific wavelengths) from the surface of an LC detection unit. In particularly preferred embodiments, plate readers are provided that are designed to be used in conjunction with LC assays. Other embodiments of the present invention provide modified commercially available readers such as ELISA readers and fluorometric readers adapted to read LC assays.

Non-limiting examples of the plate readers useful in conjunction with the present invention are provided in U.S. patent application Ser. No. 10/227,974, incorporated herein by reference. In some embodiments, two polarizing filters are placed in the optical pathway of the plate reader in a crossed or parallel polar configuration. One filter is placed on the emission side of the light path prior to passing through the sample while a second polarizing filter is placed on the analyzing side of the light path after light has passed through the sample but before it is collected by a sensing devise such as camera, a smart phone, a photodiode or a CCD. An ordered liquid crystal in the LC assay device preserves the plane of polarization and the amount of light reaching the light gathering and sensing device is markedly attenuated when viewed through cross polars or markedly accentuated when viewed through parallel polars. Random organization of the liquid crystal of the LC assay device does not preserve the plane of polarization and the amount of light, passing through crossed polars, reaching the light collecting and sensing device is relatively unaffected. Accordingly, in preferred embodiments, the binding of target molecules by the recognition moieties in an LC assay device introduces disorder into the overlying thin film of LC that increases with the amount of bound target molecule. In other embodiments, specific bandpass filters are placed on the excitation side of the light path before light encounters the sample as well as on the emission side of the light path (after light has passed through or is reflected by the sample but before reaching the light collecting and sensing device (e.g., smart phone camera, a camera, a photodiode or CCD). This configuration is useful for quantifying both reflected and transmitted light

Commercially available plate readers that may be modified according to the present invention include, but are not limited, to those available from Nalge Nunc International Corporation (Rochester, N.Y.), Greiner America, Inc. (Lake Mary, Fla.), Akers Laboratories Inc., (Thorofare, N.J.), Alpha Diagnostic International, Inc. (San Antonio, Tex.), and Qiagen Inc. (Valencia, Calif.).

### IV. Kits

In some embodiments, the present invention provides kits for the detection of biological entities comprising a lipid membrane in the respiratory droplets of a subject. In preferred embodiments, the kits comprise one or more breathing barriers configured with a liquid crystal detection device as described above. In some embodiments, the kits comprise parts to assemble a LC detection unit an in particular an LC cell. In some embodiments, the kits comprise a vial containing mesogens. In still other embodiments, the kits comprise at least one vial containing a control analyte or analytes. In still other embodiments, the kit comprises instructions for using the components contained in the kit for the detection of at least one type of analyte, preferably a biological entity comprising a lipid membrane.

In some embodiments, the instructions further comprise the statement of intended use required by the U.S. Food and Drug Administration (FDA) in labeling in vitro diagnostic products. The FDA classifies in vitro diagnostics as medical devices and requires that they be approved through the 510(k) procedure. Information required in an application under 510(k) includes: 1) The in vitro diagnostic product name, including the trade or proprietary name, the common or usual name, and the classification name of the device; 2) The intended use of the product; 3) The establishment registration number, if applicable, of the owner or operator submitting the 510(k) submission; the class in which the in vitro diagnostic product was placed under section 513 of the FD&C Act, if known, its appropriate panel, or, if the owner or operator determines that the device has not been classified under such section, a statement of that determination and the basis for the determination that the in vitro diagnostic product is not so classified; 4) Proposed labels, labeling and advertisements sufficient to describe the in vitro diagnostic product, its intended use, and directions for use. Where applicable, photographs or engineering drawings should be supplied; 5) A statement indicating that the device is similar to and/or different from other in vitro diagnostic products of comparable type in commercial distribution in the U.S., accompanied by data to support the statement; 6) A 510(k) summary of the safety and effectiveness data upon which the substantial equivalence determination is based; or a statement that the 510(k) safety and effectiveness information supporting the FDA finding of substantial equivalence will be made available to any person within 30 days of a written request; 7) A statement that the submitter believes, to the best of their knowledge, that all data and information submitted in the premarket notification are truthful and accurate and that no material fact has been omitted; 8) Any additional information regarding the in vitro diagnostic product requested that is necessary for the FDA to make a substantial equivalency determination. Additional information is available at the Internet web page of the U.S. FDA.

### EXPERIMENTAL

The following examples are provided in order to demonstrate and further illustrate certain preferred embodiments and aspects of the present invention and are not to be construed as limiting the scope thereof.

### Example 1

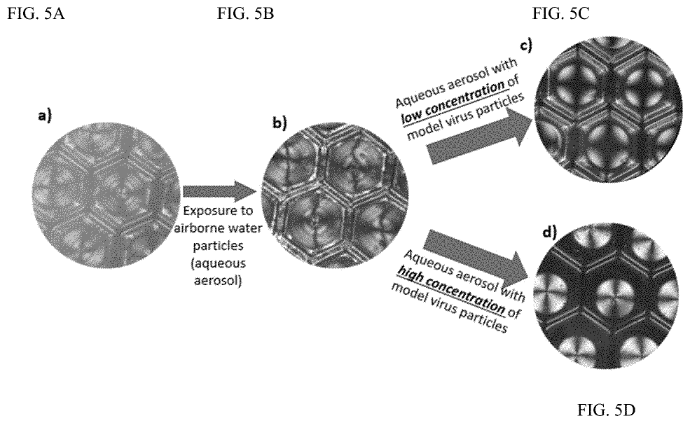

In this example, model virus particles were generated using an ultrasonic nebulizer. The model virus particles consisted of nanoparticles formed from amphiphilic molecules. LC films were exposed to high or low concentrations of the model viral particles from the nebulizer. Data is provided in FIG. 5. FIG. 5(a) is an image of LC film viewed through crossed polarized light. The LC is supported on glass surfaces patterned with hexagonal microwells. FIG. 5(b) is an image of LC film following exposure to airborne water particles (aqueous aerosol). FIG. 5(c) is an image of LC film exposed to aqueous aerosol containing a low concentration of model virus particles. The presence of model virus particles in the aerosol caused an anchoring transition in the LC film. FIG. 5(d) is an image of LC film exposed to aqueous aerosol containing high concentration of model virus particles. In this case, the high concentration of model virus particles in the aerosol caused the LC film within the microwells to de-wet and form beads at the bottom of the microwells.

### Example 2

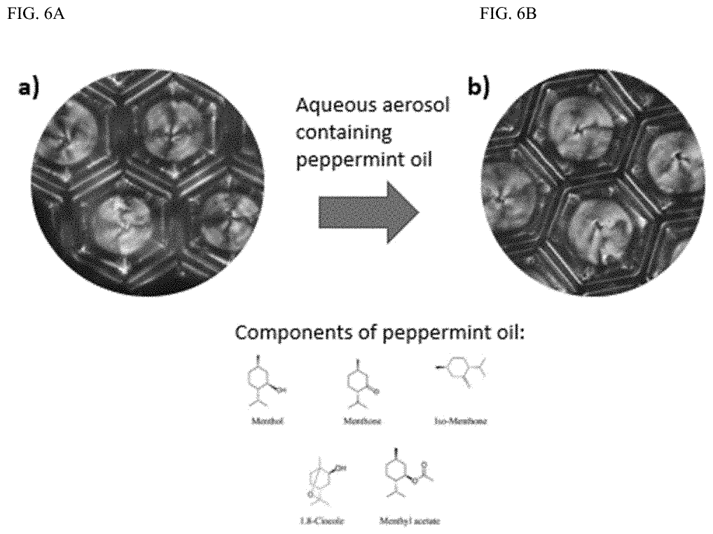

A LC film was contacted with an aqueous aerosol containing a low concentration of peppermint oil. Data is presented in FIG. 6. FIG. 6(a) is an image of LC film viewed with crossed polarized light. FIG. 6(b) is an image of LC film following exposure to aqueous aerosol containing low concentration of peppermint oil. The optical patterns of the LC film exhibit a change upon exposure to the aerosol with the oil. The significance of this experiment is that the optical texture of the LC may convey information on the molecules deposited at its surface with air. Thus, even when the molecules are not virus particles, the LC may be used to detect these other molecules.

### Example 3

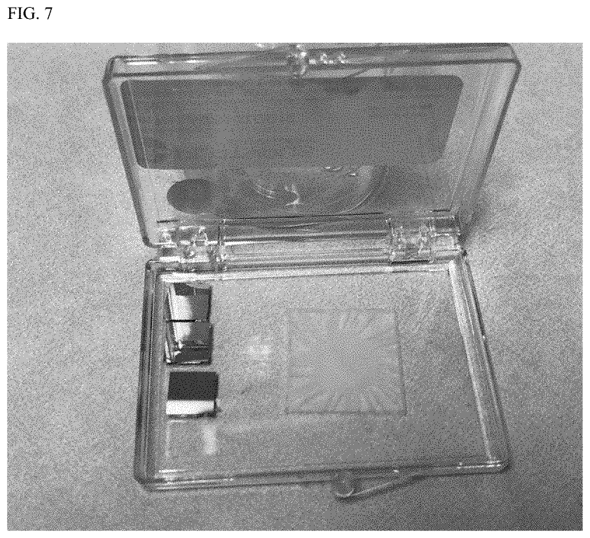

This example demonstrates detection of an enveloped virus in an aerosol. In this experiment, aerosol spray bottles were filled with one of four different solutions and used to apply an aerosol spray to a liquid crystal (LC) sensor (depicted in FIG. 7; glass square supporting an LC film positioned in case). The four solution tested were 1) pure water, 2) water with 1% w/w phospholipids, 3) virgin cell culture media, and 4) virus solution in cell culture media. The solutions were tested in LC sensors with either planar or homeotropic aligned LC as different thicknesses. The samples tested are provided in Table 2.

The samples were applied to the LC sensors as follows. The case containing the sensor was opened and supported so that the LC films were in a vertical position. The spray bottles containing the desired solutions were then placed in front of the LC film supported on the glass square so that the side of the bottle touched the edge of the case, and the nozzle was in front of the LC film. The nozzle of the spray bottle was then quickly depressed one time to deliver the aerosol into the LC film. The case were then closed, sealed with parafilm, and labelled with the solution used.

TABLE 2

| Sample # | Case # | Solution Contents | LC Alignment | LC Thickness |
|---|---|---|---|---|
| 1 | 1 | Pure Water | Planar | 25 |
| 2 | 1 | Pure Water | Planar | 50 |
| 3 | 2 | Pure Water | Homeotropic | 25 |
| 4 | 2 | Pure Water | Homeotropic | 50 |
| 5 | 3 | 1 vol. % phos. | Planar | 25 |
| 6 | 3 | 1 vol. % phos. | Planar | 50 |
| 7 | 4 | 1 vol. % phos. | Homeotropic | 25 |
| 8 | 4 | 1 vol. % phos. | Homeotropic | 50 |
| 9 | 5 | Cell culture media | Planar | 25 |
| 10 | 5 | Cell culture media | Planar | 50 |
| 11 | 6 | Cell culture media | Homeotropic | 25 |
| 12 | 6 | Cell culture media | Homeotropic | 50 |
| 13 | 7 | Virus solution | Planar | 25 |
| 14 | 7 | Virus solution | Planar | 50 |
| 15 | 8 | Virus solution | Homeotropic | 25 |
| 16 | 8 | Virus solution | Homeotropic | 50 |

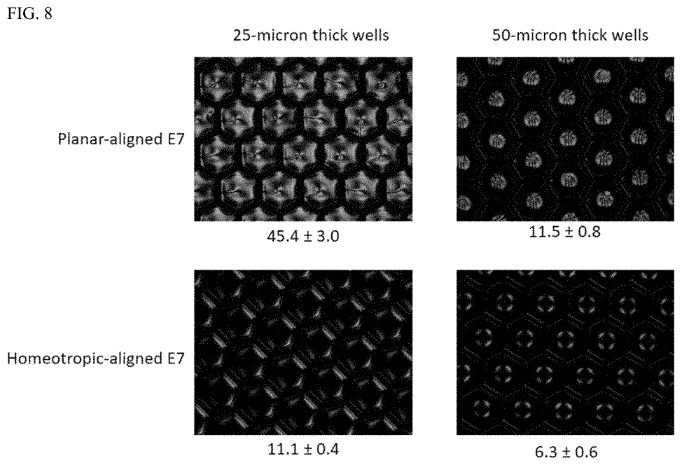

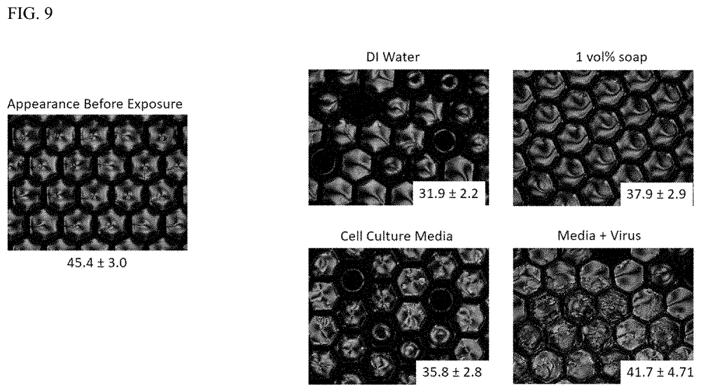

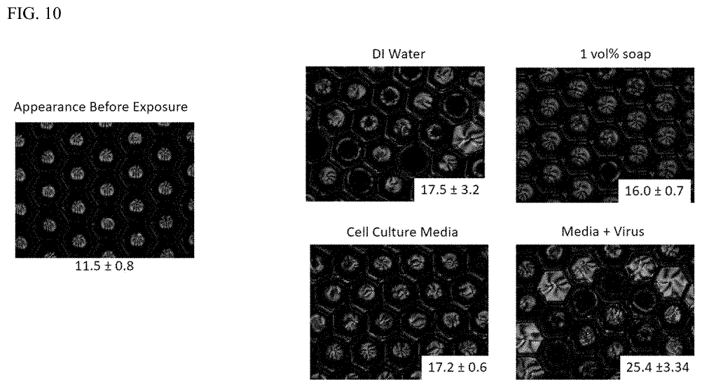

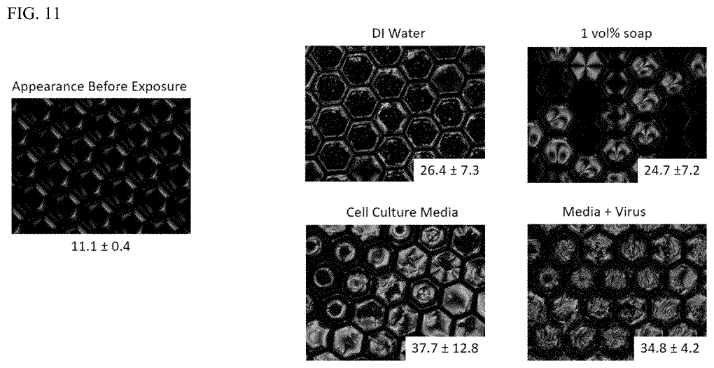

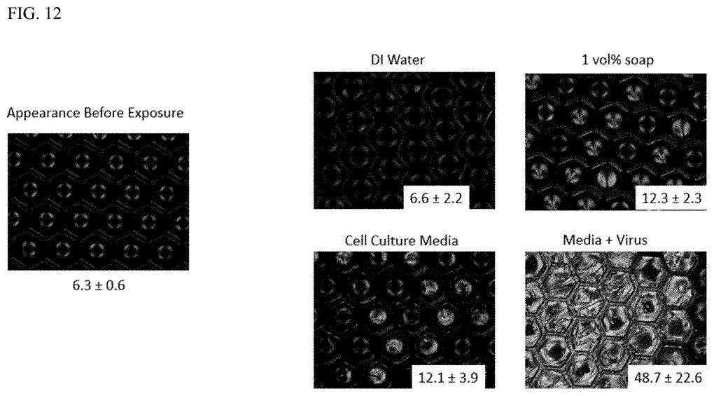

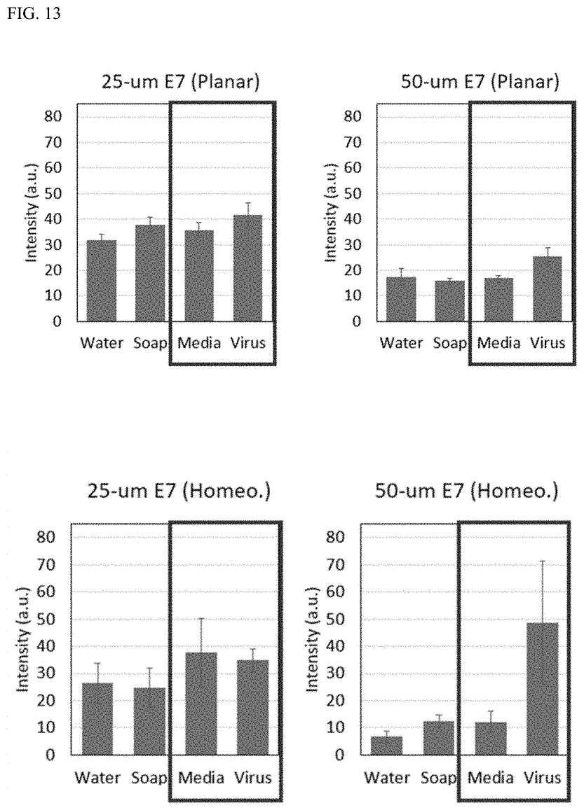

The following results were obtained. FIG. 8 provides a photograph of representative LC films before exposure. FIG. 9 provides a photograph of representative 25 μm thick E7 planar LC films 1 to 2 hours after exposure to each the four aerosol solutions. FIG. 10 provides a photograph of representative 50 μm thick E7 planar LC films 1 to 2 hours after exposure to each the four aerosol solutions. FIG. 11 provides a photograph of representative 25 μm thick E7 homeotropic LC films 1 to 2 hours after exposure to each the four aerosol solutions. FIG. 12 provides a photograph of representative 50 μm thick E7 homeotropic LC films 1 to 2 hours after exposure to each the four aerosol solutions. FIG. 13 provides a graphical summary of intensity for exposure of the different LC films to each of the four different solutions.

The following conclusions can be based on these data. From visual inspection of the images, it was observed that all LC films exposed to the virus exhibit complex features (e.g., stripped patterns). In contrast, LC films exposed to aerosols that do not contain viruses exhibit relatively smooth features. From analysis of the intensity of the images, the 50-um films of LC exhibit significantly higher brightness when exposed to media containing the virus.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in organic chemistry, materials science, chemical engineering, virology, biology, genetics, or related fields are intended to be within the scope of the following claims.

What is claimed is:

1. A method for detecting the presence of a biological entity comprising a lipid membrane in an environment inhabited by subjects comprising:
collecting air comprising aqueous aerosol particles from an environment inhabited by subjects;
contacting a liquid crystal detection unit comprising mesogens with the aqueous aerosol particles; and
monitoring the liquid crystal detection device for changes in the mesogens after or during exposure of the mesogens to the aqueous aerosol particles, wherein the change in the mesogens is indicative of the presence or absence of a biological entity comprising a lipid membrane in the aerosol particles.

2. The method of claim 1, wherein the change in the mesogens is selected from the group consisting of a change in color, a change in texture, a change in tilt, and homeotropic orientation.

3. The method of claim 1, wherein the change in mesogens is detected by a method selected from the group consisting of visual detection, optical detection, spectroscopy, light transmission, and electrical detection.

4. The method of any claim 1, wherein the biological entity comprising a lipid membrane is selected from the group consisting of mycoplasmas, bacteria and viruses.

5. The method of claim 4, wherein the virus is selected from the group consisting of the following families: Adenoviridae, Arenaviridae, Astroviridae, Birnaviridae, Bunyaviridae, Caliciviridae, Circoviridae, Coronaviridae, Filoviridae, Flaviviridae, Hepadnaviridae, Herpesviridae, Iridoviridae, Filoviridae, Orthomyxoviridae, Papovaviridae, Paramyxoviridae, Parvoviridae, Picornaviridae, Poxviridae, Reoviridae, Retroviridae, Rhabdoviridae, Togaviridae, Badnavirus, Bromoviridae, Comoviridae, Geminiviridae, Partitiviridae, Potyviridae, Sequiviridae, and Tombusviridae.

6. The method of claim 5, wherein the virus is a member of the family Coronaviridae.

7. The method of claim 6, wherein the virus is SARS-CoV-2.

8. The method of claim 5, wherein the virus is a member of the family Orthomyxoviridae.

9. The method of claim 8, wherein the virus in an influenza A or influenza B virus.

10. The method of claim 1, further comprising obtaining an image of the mesogens in the LC detection device and using an artificial intelligence algorithm to analyze the image to determine if there has been exposure to an entity comprising a biological membrane.

11. A system for detecting biological entities comprising a lipid membrane comprising:
a liquid crystal detection unit comprising mesogens disposed on the surface of a first substrate and a second substrate positioned opposite to the first substrate to form a cell having a gap between the second substrate and the mesogens disposed on the first substrate; and
an aerosol collection unit.

12. The system of claim 11, wherein the liquid crystal detection unit further comprises an adhesive backing.

13. The system of claim 12, wherein the adhesive backing is compatible with attachment of the liquid crystal detection unit to the skin.

14. The system of claim 11, wherein the aerosol collection unit is a breathing barrier is selected from the group consisting of a respirator and a mask.

15. The system of claim 11, wherein the aerosol collection unit samples air from an atmosphere.

16. The system of claim 1, wherein the mesogens are selected from the group consisting of E7, MLC, 5CB (4-n-pentyl-4′-cyanobiphenyl), 8CB (4-cyano-4′octylbiphenyl), BL093, TL 216, ZLI 5800, MLC 6613, and MBBA ((p-methoxybenzylidene)-p-butylaniline) and combinations thereof.

17. A method of detecting the presence of a biological entity comprising a lipid membrane comprising:
providing a system according to claim 11;
exposing the liquid crystal detection device to an air source suspected of containing the biological entity comprising a lipid membrane; and
monitoring the liquid crystal detection device for changes in the mesogens after or during exposure of the mesogens to the air source, wherein the change in the mesogens is indicative of the presence or absence of a biological entity comprising a lipid membrane in the air source.

18. The method of claim 17, wherein the change in the mesogens is selected from the group consisting of a change in color, a change in texture, a change in tilt, and homeotropic orientation.

19. The method of claim 17, wherein the biological entity comprising a lipid membrane is selected from the group consisting of mycoplasmas, bacteria and viruses.
